(19) United States
(12) Patent Application Publication
(10) Pub. No.: US 2023/0151046 A1
(43) Pub. Date: May 18, 2023

(54) 5-POSITION MODIFIED PYRIMIDINES

(71) Applicant: Nuclera Nucleics Ltd., Cambridge (GB)

(72) Inventors: Michael Chun Hao Chen, Cambridge (GB); Michal Robert Matuszewski, Cambridge (GB); Martin Fox, Cambridge (GB); Gordon Ross McInroy, Cambridge (GB)

(21) Appl. No.: 17/916,862

(22) PCT Filed: Apr. 6, 2021
(86) PCT No.: PCT/GB2021/050840

§ 371 Date: Oct. 4, 2022

(30) Foreign Application Priority Data
Apr. 6, 2020 (GB) 2005048.0
Oct. 9, 2020 (GB) 2016042.0

(51) Int. Cl. C07H 19/10; C07H 21/00; C12P 19/34

(52) U.S. Cl. CPC C07H 19/10; C07H 21/00; C12P 19/34

(57) ABSTRACT

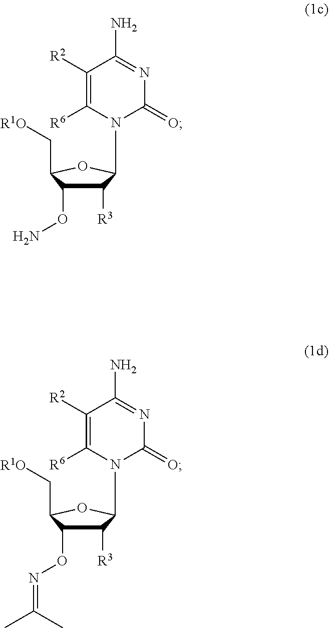

The present invention relates to a compound according to Formula (1c) or (1d): wherein, R¹; R²; R³ and R⁶ are defined herein, and their use in methods of nucleic acid synthesis.

### FIELD OF THE INVENTION

The invention relates to modified pyrimidine nucleotides having an electron withdrawing group added at the 5-position. The invention also relates to a method of nucleic acid synthesis to produce oligonucleotides containing said modified pyrimidine nucleotide. The invention further relates to a kit comprising the modified pyrimidine, a terminal transferase enzyme and optionally a salt.

### BACKGROUND TO THE INVENTION

Nucleic acid synthesis is vital to modern biotechnology. The rapid pace of development in the biotechnology arena has been made possible by the scientific community's ability to artificially synthesise DNA, RNA and proteins.

Artificial DNA synthesis allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. Despite being a mature technology, it is highly challenging to synthesise a DNA strand greater than 200 nucleotides in length in viable yield, and most DNA synthesis companies only offer up to 120 nucleotides routinely. In comparison, an average protein-coding gene is of the order of 2000-3000 contiguous nucleotides, a chromosome is at least a million contiguous nucleotides in length and an average eukaryotic genome numbers in the billions of nucleotides. In order to prepare nucleic acid strands thousands of base pairs in length, all major gene synthesis companies today rely on variations of a ‘synthesise and stitch’ technique, where overlapping 40-60-mer fragments are synthesised and stitched together by enzymatic copying and extension. Current methods generally allow up to 3 kb in length for routine production.

The reason DNA cannot be routinely synthesised beyond 120-200 nucleotides at a time is due to the current methodology for generating DNA, which uses synthetic chemistry (i.e., phosphoramidite technology) to couple a nucleotide one at a time to make DNA. Even if the efficiency of each nucleotide-coupling step is 99% efficient, it is mathematically impossible to synthesise DNA longer than 200 nucleotides in acceptable yields. The Venter Institute illustrated this laborious process by spending 4 years and 20 million USD to synthesise the relatively small genome of a bacterium.

Known methods of DNA sequencing use template-dependent DNA polymerases to add 3′-reversibly terminated nucleotides to a growing double-stranded substrate. In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

Various attempts have been made to use a terminal deoxynucleotidyl transferase for de novo single-stranded DNA synthesis. Uncontrolled de novo single stranded DNA synthesis, as opposed to controlled, takes advantage of TdT's deoxynucleoside triphosphate (dNTP) 3′ tailing properties on single-stranded DNA to create, for example, homopolymeric adaptor sequences for next-generation sequencing library preparation. In controlled extensions, a reversible deoxynucleoside triphosphate termination technology needs to be employed to prevent uncontrolled addition of dNTPs to the 3′-end of a growing DNA strand. The development of a controlled single-stranded DNA synthesis process through TdT would be invaluable to in situ DNA synthesis for gene assembly or hybridization microarrays as it removes the need for an anhydrous environment and allows the use of various polymers incompatible with organic solvents.

However, TdT has been shown not to efficiently add nucleoside triphosphates containing 3′-O-reversibly terminating moieties for building up a nascent single-stranded DNA chain necessary for a de novo synthesis cycle. A 3′-O— reversible terminating moiety would prevent a terminal transferase such as TdT from catalysing the nucleotide transferase reaction between the 3′-end of a growing DNA strand and the 5′-triphosphate of an incoming nucleoside triphosphate. The inventors have previously discovered certain modified nucleotides can be incorporated using terminal transferases. Modified nucleotides suitable for terminal transferase extension have been disclosed in for example PCT/GB2018/053305. A common reversible terminator is the aminooxy (O—NH₂) group. The aminooxy group is converted to OH by treatment with nitrite. However, the pyrimidine nucleobase cytidine carries an exocyclic NH₂ group that is also susceptible to reaction with nitrite. Reaction with nitrite leads to deamination, that is conversion of the exocyclic amine into a carbonyl. This chemical reaction introduces a mutation into the oligonucleotide, for example, deamination of cytosine into thymine is a mutation.

Pyrimidines are one of two classes of heterocyclic nitrogenous bases found in both DNA and RNA nucleic acid constructs. Pyrimidines found in DNA nitrogenous bases are cytosine (C) and thymine (T); in RNA, uracil (U) replaces thymine. These bases can form hydrogen bonds with their complementary purines—guanine (G) in the case of cytosine and adenine (A) in the case of thymine and uracil. Hydrogen bonding is of vital biochemical importance, for instance it is required to form complementary double stranded structures or select the correct tRNAs during protein translation.

Deamination changes the hydrogen bonding pattern of the base and thus alters the base pairing properties of the base. For example, cytosine is of the form donor-acceptor-acceptor (DAA) while uracil is of the form acceptor-donor-accepter (ADA). One effect of a deamination mutation is to change the efficiency with which a nucleic acid can hybridise to a target; this effect typically manifests in a decrease in the melting temperature of the duplex. A second effect of a deamination mutation is that a nucleic acid copy (for instance made by a DNA polymerase) will also contain a mutation. A third effect of a deamination mutation is to change the function of the nucleic acid, for example, by changing the amino acid sequence of a resultant peptide/protein should the nucleic acid undergo translation. The protein translated from a mutated nucleic acid would have the wrong sequence, likely fold incorrectly, and ultimately exhibit a loss of or reduction in function. Clearly, mutations are often unacceptable as they affect the properties of the nucleic acid and lead to a change in the encoded information.

### SUMMARY OF THE INVENTION

Disclosed herein a method of reducing the deamination of the cytosine base during oligonucleotide synthesis. The method is particularly applicable when nitrite is used to remove an aminooxy terminating moiety from the sugar hydroxyl.

The modified cytosine bases also provide enhanced stability during the conversion to O—NH₂ nucleotides with aminooxy compounds such as methoxylamine. For example as seen in FIG. 9, FdC is almost 10× more stable to methoxylamine treatment than canonical dC.

An aspect of the present invention relates to a compound according to Formula (1a) or (1b):

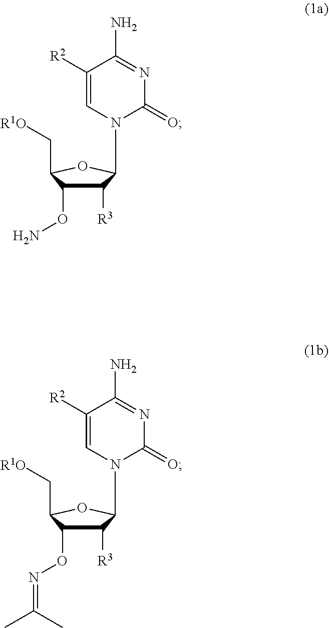

wherein,

R¹ is a phosphate or polyphosphate group or salt thereof, optionally containing one or more sulfur atoms;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and

R³ is selected from H, OH, F, OCH₃, or OCH₂CH₂OMe;

wherein R⁴ and R⁵ are independently selected from H and C₁₋₆ alkyl optionally substituted with OH or halo atoms.

A further aspect of the present invention relates to a compound according to Formula (1c) or (1d):

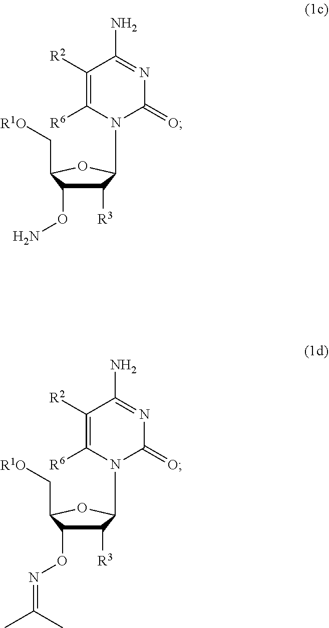

wherein,

R¹ is a phosphate or polyphosphate group or salt thereof, optionally containing one or more sulfur atoms;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵;

wherein R⁴ and R⁵ are independently selected from H and C₁₋₆ alkyl optionally substituted with OH or halo atoms; and

R⁶ is H or D.

A further aspect of the present invention relates to a method of nucleic acid synthesis comprising reacting a compound of Formula (1a) or (1b) with an oligonucleotide in the presence of a nucleic acid polymerizing enzyme, for example a DNA polymerase or terminal deoxynucleotidyl transferase (TdT) enzyme and treating the extended oligonucleotide with a nitrite salt.

A further aspect of the present invention relates to a method of nucleic acid synthesis comprising reacting a compound of Formula (1c) or (1d) with an oligonucleotide in the presence of a nucleic acid polymerizing enzyme, for example a DNA polymerase or terminal deoxynucleotidyl transferase (TdT) enzyme and treating the extended oligonucleotide with a nitrite salt.

A further aspect of the present invention relates to a kit comprising:
(i) a compound according to any one of Formula (1a) or (1b);
(ii) a terminal deoxynucleotidyl transferase (TdT) enzyme; and optionally
(iii) a nitrite salt.

A further aspect of the present invention relates to a kit comprising:
(i) a compound according to any one of Formula (1c) or (1d);
(ii) a terminal deoxynucleotidyl transferase (TdT) enzyme; and optionally
(iii) a nitrite salt.

A further aspect of the present invention relates to an oligonucleotide according to Formula (2a) or (2b):

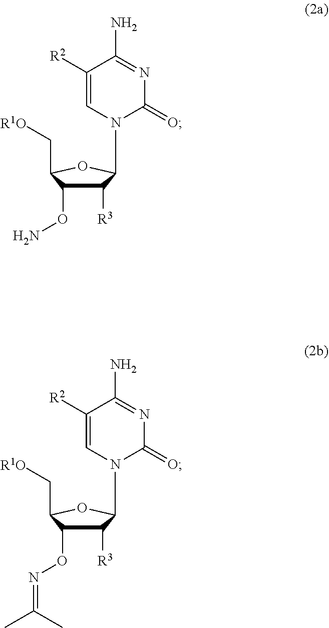

R¹ is an oligonucleotide;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and

R³ is selected from H, OH, F, OCH₃, or OCH₂CH₂OMe;

wherein R⁴ and R⁵ are independently selected from H and C₁₋₆ alkyl optionally substituted with OH or halo atoms.

A further aspect of the present invention relates to an oligonucleotide according to Formula (2c) or (2d):

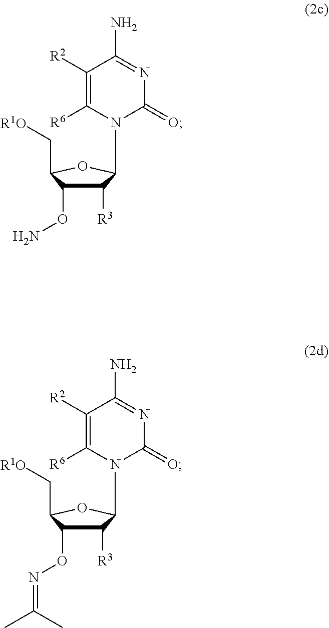

wherein,

R¹ is an oligonucleotide;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵;

R³ is selected from H, OH, F, OCH₃, or OCH₂CH₂OMe;

wherein R⁴ and R⁵ are independently selected from H and C₁₋₆ alkyl optionally substituted with OH or halo atoms; and

R⁶ is H or D.

### DETAILED DESCRIPTION OF THE INVENTION

Disclosed herein is a method of reducing the deamination of the cytosine base during oligonucleotide synthesis. The method is particularly applicable when nitrite is used to convert an aminooxy terminating moiety on the sugar to a hydroxyl. Electron withdrawing groups (EWG) in the 5-position of cytosine can dramatically reduce the nitrosative deamination of C to U. These EWG in the 5-position can increase the stability of cytosine molecules relative to the parent compound. In particular, propynyl and fluoro substituents at the 5-position decrease the rate of nitrite-mediated deamination by up to an order of magnitude. There is a significant industrial applicability because deamination changes the identity and hydrogen bonding pattern of the base, i.e. deamination introduces mutations into the product. Mutations are undesirable as they lead to change in sequence of the DNA, and thus affect the biophysical properties, biochemical properties, and information encoding properties of the DNA.

5-position modified cytidine and deoxycytidine nucleotides are of value to enzymatic DNA synthesis when using 3′-O-aminooxy reversible terminators or the precursors thereof. While deoxycytidine present in a synthesised strand will undergo a level of nitrite-mediated deamination that introduces mutations, 5-position electron withdrawing modified deoxycytidines are more robust and thus yield a higher quality product.

The 3′-O-aminooxy reversible terminator precursors may include where the aminooxy is protected as an oxime, for example N═C(CH₃)₂. The oxime can be transformed into aminooxy as part of the unblocking process. The modified cytosine bases provide enhanced stability during the conversion of O—N═C(CH₃)₂ to O—NH₂ nucleotides with aminooxy compounds such as methoxylamine.

An aspect of the present invention relates to a compound according to Formula (1a) or (1b):

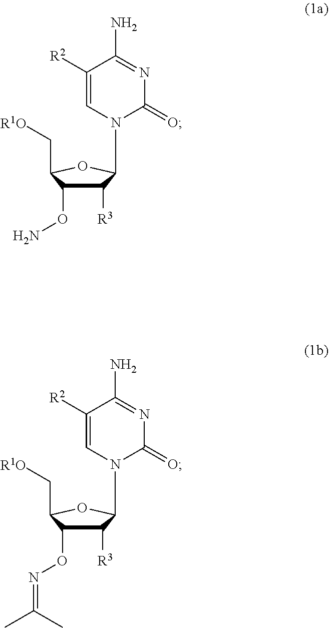

wherein,

R¹ is a phosphate or polyphosphate group or salt thereof, optionally containing one or more sulfur atoms;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and

R³ is selected from H, OH, F, OCH₃, or OCH₂CH₂OMe;

wherein R⁴ and R⁵ are independently selected from H and C₁₋₆ alkyl optionally substituted with OH or halo atoms.

An aspect of the invention involves converting compounds of Formula (1b) to compounds of Formula (1a). The conversion may be performed using aminooxy compounds. The conversion may be performed using methoxylamine. Disclosed is A method of synthesizing a compound according to formula (1a):

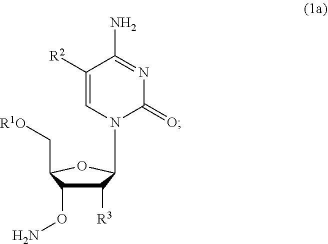

wherein,

R¹ is a phosphate or polyphosphate group or salt thereof, optionally containing one or more sulfur atoms;

R² is an electron withdrawing group (EWG) selected from the group consisting of: halo; nitrile;

halomethyl, dihalomethyl, trihalomethyl; C≡CR⁴; SOR⁴; SO₂R⁴; SO₃R⁴; COR⁴; CO₂R⁴; CONR⁴R⁵; and

R³ is selected from H, OH, F, OCH₃, or OCH₂CH₂OMe;

wherein R⁴ and R⁵ are independently selected from H and C₁₋₆ alkyl optionally substituted with OH or halo atoms comprising taking a compound according to Formula (1b):

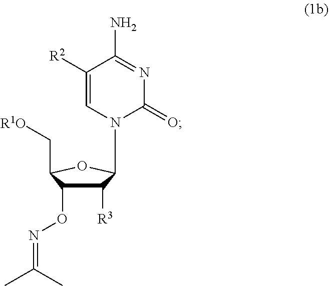

wherein,

R¹ is a phosphate or polyphosphate group or salt thereof, optionally containing one or more sulfur atoms;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and R³ is selected from H, OH, F, OCH₃, or OCH₂Ch₂OMe;

wherein R⁴ and R⁵ are independently selected from H and C₁₋₆ alkyl optionally substituted with OH or halo atoms

and treating the compounds of Formula (1b) with aminooxy compounds such as hydroxylamine, methoxylamine or ethoxylamine.

R¹ can be a phosphate or polyphosphate group. The phosphate groups can be protonated or in salt form. The phosphates can be entirely oxygen, or can contain one or more sulfur atoms. R¹ can be a phosphate group. R¹ can be a polyphosphate group. R¹ can also be a phosphate or polyphosphate group selected from —(PO₃)⁻ₓ(PO₂S)⁻_{y}(PO₃)⁻_{z} where x, y and z are independently 0-5 and x+y+z is 1-5. R¹ can also be a phosphate or polyphosphate group having one or more sulfur atoms. R¹ can be a phosphate group having one or more sulfur atoms. R¹ can be a polyphosphate group having one or more sulfur atoms. The sulfur atom can be in any position on any on the phosphate groups. R¹ can further be a monophosphate, diphosphate, triphosphate, tetraphosphate, pentaphosphate, or (alpha-thio)triphosphate group. R¹ can be a monophosphate group. R¹ can be a diphosphate group. R¹ can be a tetraphosphate group. R¹ can be a pentaphosphate group. R¹ can be an (alpha-thio)triphosphate group. R¹ can be a triphosphate group.

R² is an electron withdrawing group (EWG). R² can be an electron withdrawing group (EWG) that can be selected from the group consisting of halo, nitrile, halomethyl, dihalomethyl, trihalomethyl, C≡CR⁴, SOR⁴, SO₂R⁴, SO₃R⁴, COR⁴, CO₂R⁴ or CONR⁴R⁵. R² can be a halo group. R² can be selected from F, Cl, Br or I. R² can be a nitrile group. R² can be a halomethyl group. R² can be a dihalomethyl group. R² can be a trihalomethyl group. R² can be a C≡CR⁴ group. R² can be a SOR⁴; SO₂R⁴ or SO₃R⁴ group. R² can be an electron withdrawing group (EWG) consisting of a COR⁴ group such as an aldehyde or ketone. R² can be an electron withdrawing group (EWG) consisting of a CO₂R⁴ group such as an acid or ester. R² can be an electron withdrawing group (EWG) consisting of an amide CONR⁴R⁵ group.

R⁴ and R⁵ can be independently selected from H and C₁₋₆ alkyl optionally substituted with OH or halo atoms. R⁴ can be H. R⁴ can be C₁₋₆ alkyl optionally substituted with OH or halo atoms, wherein the halo atoms can be selected from F, Cl, Br or I. R⁴ can be CH₃. R⁴ can be CH₂OH. R⁴ can be CH₂CH₂OH.

R⁵ can be H. R⁵ can be C₁₋₆ alkyl optionally substituted with OH or halo atoms. R⁵ can be C₁₋₆ alkyl optionally substituted with OH or halo atoms, wherein the halo atoms can be selected from F, Cl, Br or I. R⁵ can be CH₃.

One embodiment of the present invention relates to a compound according to Formula (1a) or (1b) wherein R² can be selected from the group consisting of fluoro, propynyl or but-3-yn-1-ol. R² can be fluoro. R² can be propynyl. R² can be but-3-yn-1-ol.

R³ can be selected from H, OH, F, OCH₃ or OCH₂CH₂OMe. R³ can be OH. R³ can be F. R³ can be OCH₃.

R³ can be OCH₂CH₂OMe. Preferably, R³ can be H.

The compounds of Formula (1a) or (1b) can be selected from the group consisting of:

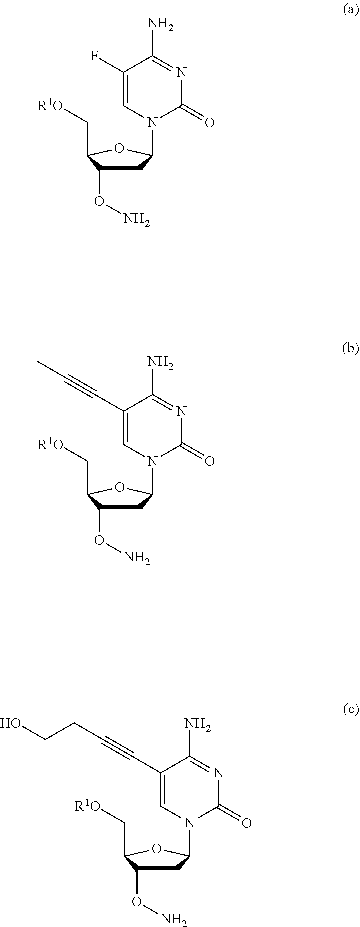

wherein, R¹ is a phosphate or polyphosphate group or salt thereof, optionally containing one or more sulfur atoms.

The compounds of Formula (1a) or (1b) can also be selected from the group consisting of:

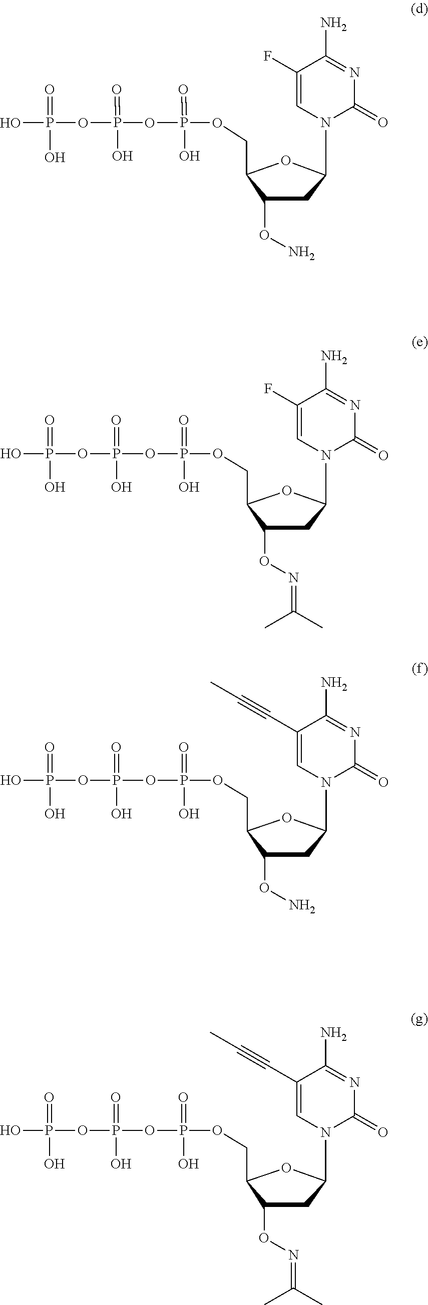 [SMILES: NO[C@@H]1C[C@H](n2cc(F)c(N)nc2=O)O[C@@H]1COP(=O)(O)OP(=O)(O)OP(=O)(O)O ; CC(C)=NO[C@@H]1C[C@H](n2cc(F)c(N)nc2=O)O[C@@H]1COP(=O)(O)OP(=O)(O)OP(=O)(O)O ; CC#Cc1cn([C@H]2C[C@@H](ON)[C@@H](COP(=O)(O)OP(=O)(O)OP(=O)(O)O)O2)c(=O)nc1N ; CC#Cc1cn([C@H]2C[C@@H](ON=C(C)C)[C@@H](COP(=O)(O)OP(=O)(O)OP(=O)(O)O)O2)c(=O)nc1N]

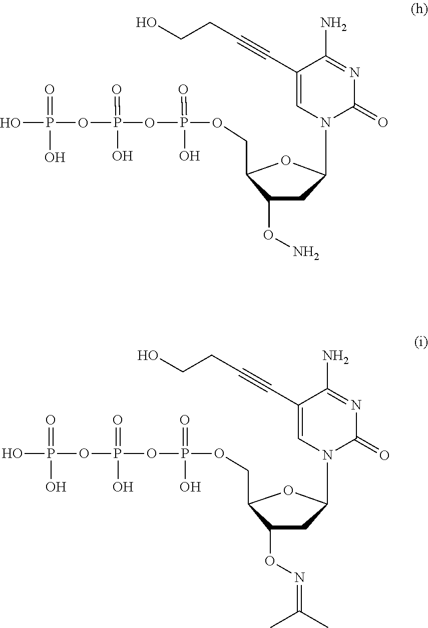 [SMILES: NO[C@@H]1C[C@H](n2cc(C#CCCO)c(N)nc2=O)O[C@@H]1COP(=O)(O)OP(=O)(O)OP(=O)(O)O ; CC(C)=NO[C@@H]1C[C@H](n2cc(C#CCCO)c(N)nc2=O)O[C@@H]1COP(=O)(O)OP(=O)(O)OP(=O)(O)O]

or salt thereof.

Included herein is a method of nucleic acid synthesis comprising reacting a compound of Formula (1a) or (1b) with an oligonucleotide in the presence of a polymerase or terminal deoxynucleotidyl transferase (TdT) enzyme and treating the extended oligonucleotide with a nitrite salt.

The terminal transferase or modified terminal transferase can be any enzyme capable of template independent strand extension. The modified terminal deoxynucleotidyl transferase (TdT) enzyme can comprise amino acid modifications when compared to a wild type sequence or a truncated version thereof. The terminal transferase can be the homologous amino acid sequence of a terminal deoxynucleotidyl transferase (TdT) enzyme in any species or the homologous amino acid sequence of Polμ, Polβ, Polλ and Polθ of any species or the homologous amino acid sequence of X family polymerases of any species.

Homologous refers to protein sequences between two or more proteins that possess a common evolutionary origin, including proteins from superfamilies in the same species of organism as well as homologous proteins from different species. Such proteins (and their encoding nucleic acids) have sequence homology, as reflected by their sequence similarity, whether in terms of percent identity or by the presence of specific residues or motifs and conserved positions. A variety of protein (and their encoding nucleic acid) sequence alignment tools may be used to determine sequence homology. For example, the Clustal Omega multiple sequence alignment program provided by the European Molecular Biology Laboratory (EMBL) can be used to determine sequence homology or homologous regions.

A further embodiment of the present invention relates to the oligonucleotide sequence comprising a solid-supported oligonucleotide sequence. The oligonucleotide sequence comprises 2 or more nucleotides. The oligonucleotide sequence can be between 10 and 500 nucleotides, such as between 20 and 200 nucleotides, in particular between 20 and 50 nucleotides long.

A further embodiment of the present invention relates to a method further comprising a reaction step with a nitrite salt. Preferably, the nitrate salt is sodium nitrite.

A further aspect of the present invention relates to a kit comprising:
(i) a compound according to any one of Formula (1a) or (1b);
(ii) a polymerase or terminal deoxynucleotidyl transferase (TdT) enzyme; and optionally
(iii) a nitrite salt.

A further aspect of the present invention relates to a compound according to Formula (1c) or (1d):

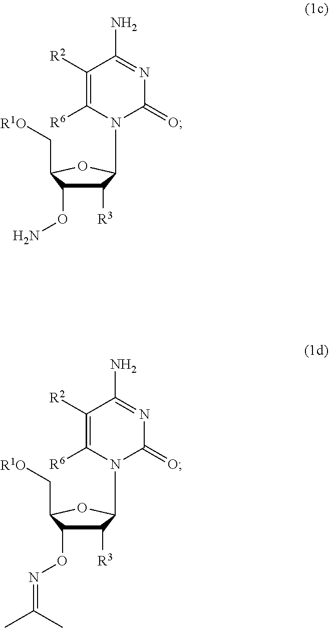

wherein,

R¹ is a phosphate or polyphosphate group or salt thereof, optionally containing one or more sulfur atoms;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; R³ is selected from H, OH, F, OCH₃, or OCH₂CH₂OMe;

wherein R⁴ and R⁵ are independently selected from H and C₁₋₆ alkyl optionally substituted with OH or halo atoms; and

R⁶ is H or D.

R¹ can be a phosphate or polyphosphate group. The phosphate groups can be protonated or in salt form. The phosphates can be entirely oxygen, or can contain one or more sulfur atoms. R¹ can be a phosphate group. R¹ can be a polyphosphate group. R¹ can also be a phosphate or polyphosphate group selected from —(PO₃)⁻ₓ(PO₂S)⁻_{y}(PO₃)⁻_{z} where x, y and z are independently 0-5 and x+y+z is 1-5. R¹ can also be a phosphate or polyphosphate group having one or more sulfur atoms. R¹ can be a phosphate group having one or more sulfur atoms. R¹ can be a polyphosphate group having one or more sulfur atoms. The sulfur atom can be in any position on any on the phosphate groups. R¹ can further be a monophosphate, diphosphate, triphosphate, tetraphosphate, pentaphosphate, or (alpha-thio)triphosphate group. R¹ can be a monophosphate group. R¹ can be a diphosphate group. R¹ can be a tetraphosphate group. R¹ can be a pentaphosphate group. R¹ can be an (alpha-thio)triphosphate group. R¹ can be a triphosphate group.

R² is an electron withdrawing group (EWG). R² can be an electron withdrawing group (EWG) that can be selected from the group consisting of halo, nitrile, halomethyl, dihalomethyl, trihalomethyl, C≡CR⁴, SOR⁴, SO₂R⁴, SO₃R⁴, COR⁴, CO₂R⁴ or CONR⁴R⁵. R² can be a halo group. R² can be selected from F, Cl, Br or I. R² can be a nitrile group. R² can be a halomethyl group. R² can be a dihalomethyl group. R² can be a trihalomethyl group. R² can be a C≡CR⁴ group. R² can be a SOR⁴; SO₂R⁴ or SO₃R⁴ group. R² can be an electron withdrawing group (EWG) consisting of a COR⁴ group such as an aldehyde or ketone. R² can be an electron withdrawing group (EWG) consisting of a CO₂R⁴ group such as an acid or ester. R² can be an electron withdrawing group (EWG) consisting of an amide CONR⁴R⁵ group.

R⁴ and R⁵ can be independently selected from H and C₁₋₆ alkyl optionally substituted with OH or halo atoms. R⁴ can be H. R⁴ can be C₁₋₆ alkyl optionally substituted with OH or halo atoms, wherein the halo atoms can be selected from F, Cl, Br or I. R⁴ can be CH₃. R⁴ can be CH₂OH. R⁴ can be CH₂CH₂OH.

R⁵ can be H. R⁵ can be C₁₋₆ alkyl optionally substituted with OH or halo atoms. R⁵ can be C₁₋₆ alkyl optionally substituted with OH or halo atoms, wherein the halo atoms can be selected from F, Cl, Br or I. R⁵ can be CH₃.

R³ can be selected from H, OH, F, OCH₃ or OCH₂CH₂OMe. R³ can be OH. R³ can be F. R³ can be OCH₃. R³ can be OCH₂CH₂OMe. Preferably, R³ can be H.

R⁶ can be selected from H or D. R⁶ can be H. R⁶ can be D.

A further aspect of the present invention relates to an oligonucleotide according to Formula (2a) or (2b):

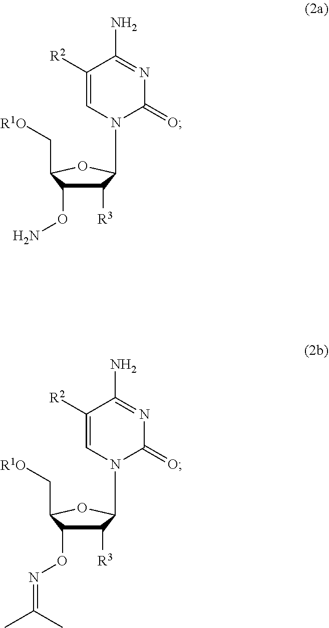

wherein,

R¹ is an oligonucleotide;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and

R³ is selected from H, OH, F, or OCH₃;

wherein R⁴ and R⁵ are independently selected from H, OH, and C₁₋₆ alkyl optionally substituted with OH or halo atoms.

A further embodiment of the present invention relates to an oligonucleotide according to Formula (2a) or (2b) wherein R¹ can be an oligonucleotide. The phosphates in R¹ can contain one or more sulfur atoms.

A further embodiment of the present invention relates to a compound according to Formula (2a) or (2b) wherein R² can be an electron withdrawing group (EWG). R² can be an electron withdrawing group (EWG) that can be selected from the group consisting of halo, nitrile, halomethyl, dihalomethyl, trihalomethyl, C≡CR⁴, SOR⁴, SO₂R⁴, SO₃R⁴, COR⁴, CO₂R⁴ or CONR⁴R⁵. R² can be an electron withdrawing group (EWG) consisting of a halo group. R² can be an electron withdrawing group (EWG) consisting of a halo group which can be selected from F, Cl, Br or I. R² can be an electron withdrawing group (EWG) consisting of a nitrile group. R² can be an electron withdrawing group (EWG) consisting of a halomethyl group. R² can be an electron withdrawing group (EWG) consisting of a dihalomethyl group. R² can be an electron withdrawing group (EWG) consisting of a trihalomethyl group. R² can be an electron withdrawing group (EWG) consisting of a C≡CR⁴ group. R² can be an electron withdrawing group (EWG) consisting of a SOR⁴ group. R² can be an electron withdrawing group (EWG) consisting of a SO₂R⁴ group. R² can be an electron withdrawing group (EWG) consisting of a COR⁴ group. R² can be an electron withdrawing group (EWG) consisting of a CO₂R⁴ group. R² can be an electron withdrawing group (EWG) consisting of a CONR⁴R⁵ group.

A further embodiment of the present invention relates to an oligonucleotide according to Formula (2a) or (2b) wherein R³ can be selected from H, OH, F, or OCH₃. R³ can be OH. R³ can be F. R³ can be OCH₃. Preferably, R³ can be H.

A further embodiment of the present invention relates to a compound according to Formula (2a) or (2b) wherein R⁴ can be independently selected from H, OH, and C₁₋₆ alkyl optionally substituted with OH or halo atoms. R⁴ can be H. R⁴ can be OH. R⁴ can be C₁₋₆ alkyl optionally substituted with OH or halo atoms, wherein the halo atoms can be selected from F, Cl, Br or I.

A further embodiment of the present invention relates to a compound according to Formula (2a) or (2b) wherein R⁵ can be independently selected from H, OH, and C₁₋₆ alkyl optionally substituted with OH or halo atoms. R⁵ can be H. R⁵ can be OH. R⁵ can be C₁₋₆ alkyl optionally substituted with OH or halo atoms, wherein the halo atoms can be selected from F, Cl, Br or I.

A further aspect of the present invention relates to an oligonucleotide according to Formula (2c) or (2d):

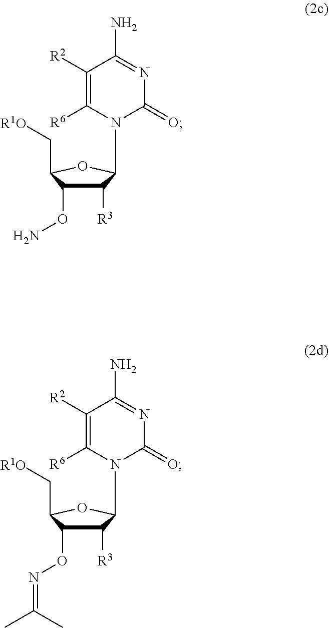

wherein,

R¹ is an oligonucleotide;

R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵;

R³ is selected from H, OH, F, OCH₃, or OCH₂CH₂OMe;

wherein R⁴ and R⁵ are independently selected from H and C₁₋₆ alkyl optionally substituted with OH or halo atoms; and

R⁶ is H or D.

A further embodiment of the present invention relates to an oligonucleotide according to Formula (2c) or (2d) wherein R¹ can be an oligonucleotide. The phosphates in R¹ can contain one or more sulfur atoms.

A further embodiment of the present invention relates to a compound according to Formula (2c) or (2d) wherein R² can be an electron withdrawing group (EWG). R² can be an electron withdrawing group (EWG) that can be selected from the group consisting of halo, nitrile, halomethyl, dihalomethyl, trihalomethyl, C≡CR⁴, SOR⁴, SO₂R⁴, SO₃R⁴, COR⁴, CO₂R⁴ or CONR⁴R⁵. R² can be an electron withdrawing group (EWG) consisting of a halo group. R² can be an electron withdrawing group (EWG) consisting of a halo group which can be selected from F, Cl, Br or I. R² can be an electron withdrawing group (EWG) consisting of a nitrile group. R² can be an electron withdrawing group (EWG) consisting of a halomethyl group. R² can be an electron withdrawing group (EWG) consisting of a dihalomethyl group. R² can be an electron withdrawing group (EWG) consisting of a trihalomethyl group. R² can be an electron withdrawing group (EWG) consisting of a C≡CR⁴ group. R² can be an electron withdrawing group (EWG) consisting of a SOR⁴ group. R² can be an electron withdrawing group (EWG) consisting of a SO₂R⁴ group. R² can be an electron withdrawing group (EWG) consisting of a COR⁴ group. R² can be an electron withdrawing group (EWG) consisting of a CO₂R⁴ group. R² can be an electron withdrawing group (EWG) consisting of a CONR⁴R⁵ group.

A further embodiment of the present invention relates to an oligonucleotide according to Formula (2c) or (2d) wherein R³ can be selected from H, OH, F, or OCH₃. R³ can be OH. R³ can be F. R³ can be OCH₃. Preferably, R³ can be H.

A further embodiment of the present invention relates to a compound according to Formula (2c) or (2d) wherein R⁴ can be independently selected from H, OH, and C₁₋₆ alkyl optionally substituted with OH or halo atoms. R⁴ can be H. R⁴ can be OH. R⁴ can be C₁₋₆ alkyl optionally substituted with OH or halo atoms, wherein the halo atoms can be selected from F, Cl, Br or I.

A further embodiment of the present invention relates to a compound according to Formula (2c) or (2d) wherein R⁵ can be independently selected from H, OH, and C₁₋₆ alkyl optionally substituted with OH or halo atoms. R⁵ can be H. R⁵ can be OH. R⁵ can be C₁₋₆ alkyl optionally substituted with OH or halo atoms, wherein the halo atoms can be selected from F, Cl, Br or I.

A further embodiment of the present invention relates to a compound according to Formula (2c) or (2d) wherein R⁶ can be selected from H or D. R⁶ can be H. R⁶ can be D.

Described herein is a process of nucleic acid synthesis using the compounds described herein. The process uses a nucleic acid polymerase, which may be a template independent polymerase or a template dependent polymerase to add a single nucleotide to one or more nucleic acid strands. The strands may be immobilised on a solid support. The process involves cleaving the 3′-aminooxy group and adding a further nucleotide, the base of which may or may not be C.

Disclosed is a method of nucleic acid synthesis comprising:
(a) providing an initiator sequence;
(b) adding extension reagents comprising a polymerase or terminal deoxynucleotidyl transferase (TdT) and a compounds according to Formula (1a) or (1b):

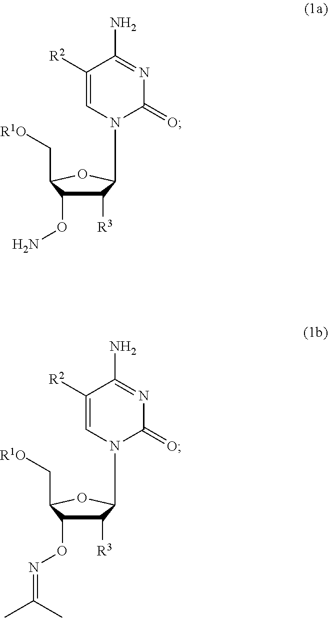

to said initiator sequence to add a single nucleotide to the initiator sequence, wherein,
R¹ is a phosphate or polyphosphate group or salt thereof, optionally containing one or more sulfur atoms;
R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and
R³ is selected from H, OH, F, OCH₃, or OCH₂CH₂OMe;
wherein R⁴ and R⁵ are independently selected from H and C₁₋₆ alkyl optionally substituted with OH or halo atoms;
(c) removal of the extension reagents;
(d) optionally transforming the N═C(CH₃)₂ if present to NH₂;
(e) cleaving the 3′-O—NH₂ group from the extended nucleic acid polymer;
(f) adding extension reagents comprising a 3′-O—NH₂ or 3′-O—N═C(CH₃)₂ blocked nucleoside triphosphate and a polymerase or terminal deoxynucleotidyl transferase (TdT) to said initiator sequence to add a further single nucleotide to the initiator sequence.

The nucleic acids synthesised can be any sequence. One or more, possibly all, of the cytosine bases will have the electron withdrawing group at the 5-position. A population of different sequences can be synthesised in parallel.

Where the other heterocyclic bases have exocyclic NH₂ groups, for example adenine or guanine, these groups can optionally be masked by an orthogonal masking agent. The amine masked nitrogenous heterocycles may be N6-amine masked adenine and N2-amine masked guanine. The masking may be for example an azido (N₃) group. Example for suitable masking groups include azide (—N₃), benzoylamine (N- benzoyl or —NHCOPh), N-methyl (—NHMe), isobutyrylamine, dimethylformamidylamine, 9-fluorenylmethyl carbamate, t-butyl carbamate, benzyl carbamate, acetamide (N-acetyl or —NHCOMe), trifluoroacetamide, pthlamide, benzylamine (N-benzyl or —NH—CH₂-phenyl), triphenylmethylamine, benxylideneamine, tosylamide, isothiocyanate, N-allyl (such as N-dimethylallyl (—NHCH₂—CH═CH₂)) and N-anisoyl (—NHCOPh-OMe), such as azide (—N₃), N- acetyl (—NHCOMe), N-benzyl (—NH—CH₂-phenyl), N-anisoyl (—NHCOPh-OMe), N-methyl, (—NHMe), N-benzoyl (—NHCOPh), N-dimethylallyl (—NHCH₂—CH═CH₂).

References herein to an “amine masking group” refer to any chemical group which is capable of generating or “unmasking” an amine group which is involved in hydrogen bond base-pairing with a complementary base. Most typically the unmasking will follow a chemical reaction, most suitably a simple, single step chemical reaction. The amine masking group will generally be orthogonal to the 3′-O—NH₂ blocking group in order to allow selective removal.

In the nucleic acids synthesised, the bases can be selected from: T or modified T such as for example ‘super-T’; C or a modified C such as for example a C having an electron withdrawing group at the 5 position, as described herein; A or a modified A such as for example an N6-amine masked adenine; and G or a modified G such as for example an N2-amine masked guanine. The amino masking group prevents de-amination caused by the nitrite exposure needed to remove the O—NH₂ at the 3′-position of the sugar.

The T nucleotides can be selected from

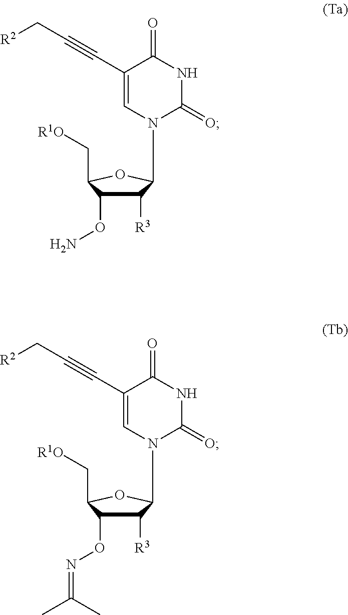

wherein, R¹ is a phosphate or polyphosphate group or salt thereof, optionally containing one or more sulfur atoms;

R² is H, halo, OH, NH₂, COOH, COH, C₁₋₃ alkoxy, C₁₋₃ alkyl optionally substituted with OH, NH₂ or halo atoms; and

R³ is selected from H, OH, F, OCH₃ or OCH₂CH₂OMe.

The T nucleotides can be

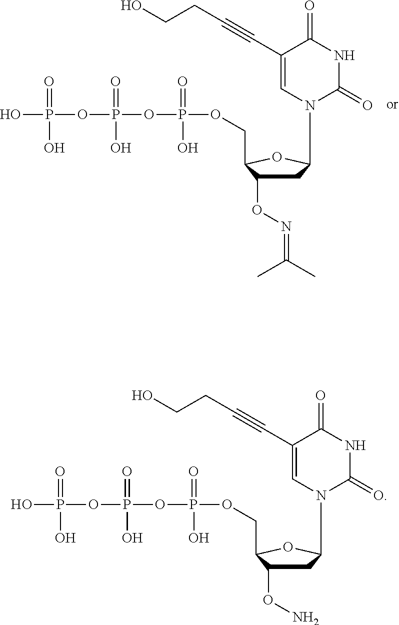

or a salt thereof.

The purine compounds may be selected from:

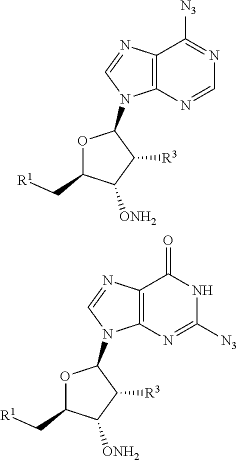

where R¹ and R³ are as defined herein.

The term ‘azide’ or ‘azido’ used herein refers to an —N₃, or more specifically, an —N═N⁺═N⁻ group. It will also be appreciated that azide extends to the presence of a tetrazolyl moiety. The “azide-tetrazole” equilibrium is well known to the skilled person from Lakshman et al (2010) J. Org. Chem. 75, 2461-2473. Thus, references herein to azide extend equally to tetrazole as illustrated below when applied to the R³ groups defined herein:

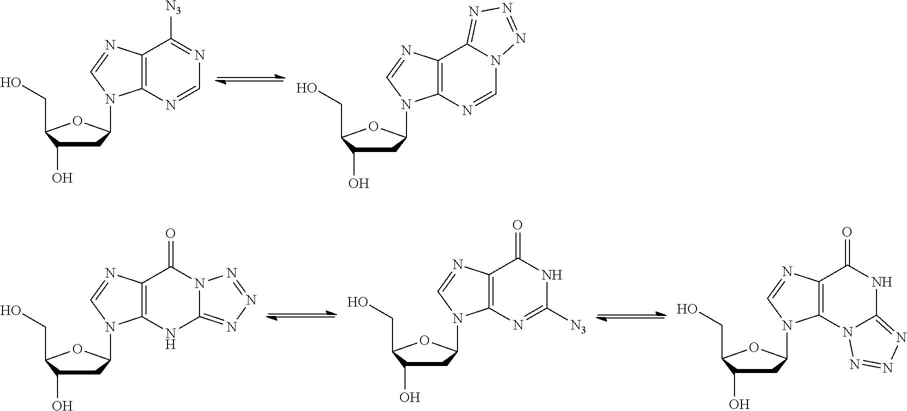

This embodiment has the advantage of reversibly masking the —NH₂ group. While blocked in the —N₃ state, the base (B) is impervious to deamination (e.g., deamination in the presence of sodium nitrite). The base (B) in the N-blocked form is incapable of forming secondary structures via base pairing. Thus, even blocking a subset of the free amino groups in the nucleic acid polymer improves the availability of the 3′-end for further extension. The canonical adenine or guanine can be respectively recovered from 6-azido adenine and 2-azido guanine by exposure to a reducing agent (e.g., TCEP). Thus, the —N₃ group serves as an effective protecting group against deamination, especially in the presence of sodium nitrite.

It will be appreciated that the compounds of the invention may be readily applied to methods of enzymatic nucleic acid synthesis which are well known to the person skilled in the art. Non-limiting methods of nucleic acid synthesis may be found in WO 2016/128731, WO 2016/139477, WO 2017/009663, GB 1613185.6 and GB 1714827.1, the contents of each of which are herein incorporated by reference.

Enzymatic nucleic acid synthesis is defined as any process in which a nucleotide is added to a nucleic acid strand through enzymatic catalysis in the presence or absence of a template. For example, a method of enzymatic nucleic acid synthesis could include non-templated de novo nucleic acid synthesis utilizing a PoIX family polymerase, such as terminal deoxynucleotidyl transferase, and reversibly terminated 2′-deoxynucleoside 5′-triphosphates or ribonucleoside 5′-triphosphate. Another method of enzymatic nucleic acid synthesis could include templated nucleic acid synthesis, including sequencing-by-synthesis. Reversibly terminated enzymatic nucleic acid synthesis is defined as any process in which a reversibly terminated nucleotide is added to a nucleic acid strand through enzymatic catalysis in the presence or absence of a template. Thus, in one embodiment, the method of enzymatic nucleic acid synthesis is selected from a method of reversibly terminated enzymatic nucleic acid synthesis and a method of templated and non-templated de novo enzymatic nucleic acid synthesis.

References herein to ‘nucleoside triphosphates’ refer to a molecule containing a nucleoside (i.e. a base attached to a deoxyribose or ribose sugar molecule) bound to three phosphate groups. Examples of nucleoside triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleoside triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleoside triphosphates, such as naturally occurring modified nucleosides and artificial/modified/non-naturally occurring nucleosides.

Therefore, references herein to ‘3’-blocked nucleoside triphosphates' refer to nucleoside triphosphates (e.g., dATP, dGTP, dCTP or dTTP) which have an additional group on the 3′-end which prevents further addition of nucleotides, i.e., by replacing the 3′-OH group with a protecting group. Herein the protecting group is NH₂ or a protected version thereof.

References herein to a ‘DNA initiator sequence’ refer to a small sequence of DNA which the 3′-blocked nucleoside triphosphate can be attached to, i.e., DNA will be synthesised from the 3′-end of the DNA initiator sequence.

In one embodiment, the initiator sequence is between 5 and 100 nucleotides long, such as between 10 and 60 nucleotides long, in particular between 20 and 50 nucleotides long. The ideal length of initiator may be informed by the immobilisation state (i.e. in solution or immobilised), the immobilisation chemistry, the initiator base sequence, and other parameters.

In one embodiment, the initiator sequence is single-stranded. In an alternative embodiment, the initiator sequence is double-stranded. In a further embodiment, the initiator sequence has double-stranded and single-stranded portions. It will be understood by persons skilled in the art that a 3′-overhang (i.e., a free 3′-end) allows for efficient addition.

In one embodiment, the initiator sequence is immobilised on a solid support. This allows the enzyme and the cleaving agent to be removed without washing away the synthesised nucleic acid. The initiator sequence may be attached to a solid support stable under aqueous conditions so that the method can be easily performed via a flow setup.

In one embodiment, the initiator sequence is immobilised on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In one embodiment, the initiator sequence contains a base or base sequence recognisable by an enzyme. A base recognised by an enzyme, such as a glycosylase, may be removed to generate an abasic site which may be cleaved by chemical or enzymatic means. An example of such a glycosylase system includes the presence of a uracil base in the initiator sequence, which may be excised with uracil DNA glycosylase (UDG) to leave an abasic site which may be cleaved with, for example, basic solutions, organic amines, or an endonuclease (such as endonuclease VIII), to release a nucleic acid bearing a 5′-phosphate into solution. A base sequence may be recognised and cleaved by a restriction enzyme.

In a further embodiment, the initiator sequence is immobilised on a solid support via an orthogonal chemically-cleavable linker, such as a disulfide, allyl, or azide-masked hemiaminal ether linker. Therefore, in one embodiment, where an azido N-masking group is not present, the method additionally comprises extracting the resultant nucleic acid by cleaving the chemical linker through the addition of tris(2-carboxyethyl)phosphine (TCEP) or dithiothreitol (DTT) for a disulfide linker;

palladium complexes or an allyl linker; or TCEP for an azide-masked hemiaminal ether linker.

In one embodiment, the resultant nucleic acid is extracted and amplified by polymerase chain reaction (PCR) using the nucleic acid bound to the solid support as a template. The initiator sequence could therefore contain an appropriate forward primer sequence and an appropriate reverse primer could be synthesised or incorporated via ligation.

In one embodiment, the terminal deoxynucleotidyl transferase (TdT) of the invention is added in the presence of an extension solution comprising one or more buffers (e.g., Tris or cacodylate), one or more salts (e.g., Na⁺, K⁺, mg²⁺, Mn²⁺, Cu²⁺, Zn²⁺, Co²⁺, etc. all with appropriate counterions, such as CI) and inorganic pyrophosphatase (e.g., the Saccharomyces cerevisiae homolog). It will be understood that the choice of buffers and salts depends on the optimal enzyme activity and stability. The use of an inorganic pyrophosphatase helps to reduce the build-up of pyrophosphate due to nucleoside triphosphate hydrolysis by TdT. Therefore, the use of an inorganic pyrophosphatase has the advantage of reducing the rate of (1) backwards reaction and (2) TdT strand dismutation.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

The compounds of the invention can be used on a device for nucleic acid synthesis. In one embodiment of the invention there is a solid support in the form of for example a planar array and further a plurality of beads onto which a plurality of immobilized initiation oligonucleotide sequences are attached. The beads may be porous and a portion of the, optionally porous, beads are selected as anchors and unselected beads are exposed to harvest solution to cleave them from their solid support to release the oligonucleotide sequences into solution. Thus the term solid support can refer to an array having a plurality of beads which may or may not be immobilised. The oligonucleotides may be attached to, or removed from beads whilst on the array. Thus the immobilised oligonucleotide may be attached to a bead, which remains in a fixed position on the array whilst other beads in other locations are subject to cleavage conditions to detach the oligonucleotides from the beads (the beads may or may not be immobilised).

The solid support can take the form of a digital microfluidic device. Digital microfluidic devices consist of a plurality of electrodes arranged on a surface. A dielectric layer (e.g., aluminum oxide) is deposited over the electrodes followed by a hydrophobic coating (e.g., perfluorinated hydrocarbon polymer) atop the dielectric layer. The electrodes may be hardwired or formed from an active matrix thin film transistor (AM-TFT).

The solid support can take the form of a digital microfluidic device. Digital microfluidic devices consist of a plurality of electrodes arranged on a surface. These electrodes can be addressed in a passive manner or by active matrix methods. Passive addressing is a direct address where actuation signals are directly applied on individual electrode (for example by means of a hard-wired connection to that electrode in a single layer or multilayer fashion such as a printed circuit board, PCB). However, a limitation of direct drive methods is the inability to process large numbers of droplets due to difficulties in addressing large numbers of direct drive electrodes. In active matrix addressing, M×N electrodes can be controlled by M+N pins, significantly reducing the number of control pins. However, the resolution of the electrodes (size of electrodes as compared to the size of droplets) limits the scope of droplet operations. Active matrix thin film transistor (AM-TFT) technology enables the control of large numbers of droplets by replacing patterned electrodes with a thin film transistor array, each of which is individually addressable. The increased resolution (small size of pixels on the thin film transistor array) also increases the scope of droplet operations. An AM-TFT digital microfluidic device comprises a dielectric layer (e.g., aluminum oxide) deposited over the electrode layer on the thin-film transistor layer followed by a hydrophobic coating (e.g., perfluorinated hydrocarbon polymer) atop the dielectric layer.

Depending on applied voltage to a subset of the plurality of electrodes arranged on the aforementioned surface, aqueous droplets may be actuated across the surface immersed in oil, air, or another fluid. Enzymatic oligonucleotide synthesis can be deployed on a digital microfluidic device in several ways. An initiator oligonucleotide can be immobilized via the 5′-end on super paramagnetic beads or directly to the hydrophobic surface of the digital microfluidic device. A plurality of distinct positions containing immobilized initiator oligonucleotides on the digital microfluidic device may be present (henceforth named synthesis zones). Solutions required for enzymatic oligonucleotide synthesis are then dispensed from multiple reservoirs onto the device. Briefly, an addition solution containing the components necessary for the TdT-mediated incorporation of reversibly terminated nucleoside 5′-triphosphates onto immobilized initiator oligonucleotides can be dispensed from a reservoir in droplets and actuated to the aforementioned positions containing immobilized initiator oligonucleotides. During this stage, each reservoir (and thus each droplet containing addition solution) can contain a distinct nitrogenous base reversibly terminated nucleoside 5′-triphosphate identity or a mixture thereof in order to control the sequence synthesized on aforementioned positions containing immobilized initiator oligonucleotides.

Alternatively the method can be implemented on continuous flow microfluidic devices. One such device consists of a surface with a plurality of microwells each containing a bead. On said bead, an oligonucleotide initiator can be immobilized. In addition to each microwell containing a bead with immobilized initiator, each microwell can contain an electrode to perform electrochemistry. Another implementation of continuous flow microfluidics consists of a fritted column containing beads or resin on which initiator sequences are immobilized. Addition, wash, and deblocking solutions may be sequentially flowed through the column in a process of DNA synthesis.

In all examples of nucleic acid synthesis, the use of the modified cytosine bases having the electron withdrawing groups improves the quality of the synthesised strands due to lowering the level of deamination.

### Examples

### 5-fluoro-2′-deoxycytidine undergoes nitrite-mediated deamination at a rate 4-fold lower than the canonical deoxycytidine analogue (FIG. 1)

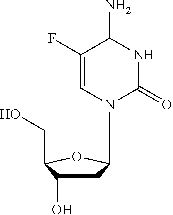

### 5-methyl-2′deoxycytidine undergoes nitrite-mediated deamination at a rate 2-fold greater than the canonical deoxycytidine analogue (FIG. 2)

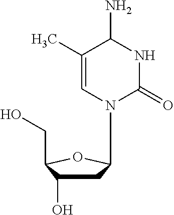

A series of 5-position modified nucleosides were tested. Each was incubated at room temperature in 700 mM NaNO₂, 1 M acetate buffer pH 5.5 and the stability assayed by LC-MS over a time course. Analysis shows that substituents can either increase or decrease the stability to nitrite solution. Electron withdrawing substituents such as the fluoro or alkynyl substituents increase stability. Electron donating substituents such as methyl and hydroxymethyl decrease stability.

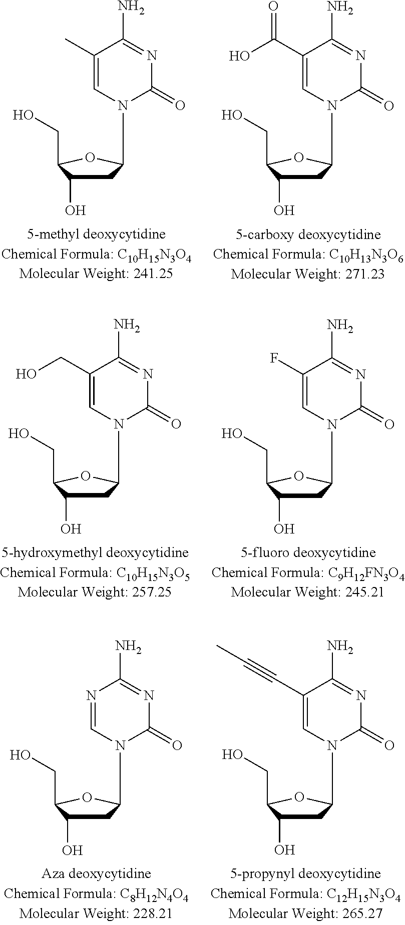

LC-MS was used to monitor nucleosides throughout a time course of incubation in 700 mM sodium nitrite at pH 5.5. Integration of UV chromatograms was performed, and the percentage of starting nucleoside plotted. Nucleosides that are more stable to the nitrite treatment will retain values closer to 100% of starting nucleotide integration (FIG. 3).

To clearly show the difference in stability between 2′-deoxycytidine, 5-carboxy-2′-deoxycytidine, 5-propynyl-2′-deoxycytidine, and 5-hydroxymethyl-2′-deoxycytidine, the plot is presented (FIG. 4) without the inclusion of 5-aza-2′-deoxycytidine.

In addition to monitoring the persistence of the starting material UV peak, the deamination products can be monitored. Deamination products were identified by their mass signals and appearance under nitrite incubation (FIG. 5).

For some particularly stable nucleosides, the time course was extended. After 6 days of incubation in the nitrite solution, only 2.5% of the starting nucleoside had been deaminated (FIG. 6).

Stability of 2′-Deoxycytidines in NDS at r.t. Rate plot of degradation in the presence of nitrite solution for a set of the substituents. 5-propynyl shows increased stability relative to the parent compound deoxycytidine, 5-carboxy deoxycytidine, and 5-hydroxymethyl deoxycytidine. The time course plots were normalised vs the deoxycytidine control run alongside each experiment (FIGS. 7&8).

Overall, propynyl and fluoro substituents at the 5-position decrease the rate of nitrite-mediated deamination by up to an order of magnitude. There is significant industrial applicability because deamination changes the hydrogen bonding pattern of the base and this introduces mutations into the product. Mutations are unacceptable as they lead to a change in information encoded in the DNA; for example, a protein translated from mutated DNA would have the wrong sequence, likely fold incorrectly, and ultimately exhibit a loss of or reduction in function.

5-position modified cytidine and deoxycytidine nucleotides are of value to enzymatic DNA synthesis processing using 3′-O-aminooxy reversible terminators. While deoxycytidine present in a synthesised strand will undergo nitrite-mediated deamination that introduces mutations, 5-position electron withdrawing modified deoxycytidines can be 10-fold more robust and thus yield a higher quality product.

Stability Test of 2′-Deoxycytidine and 2′-Deoxy-5-Fluorocytidine to methoxylamine Oxime removal solution buffer (2× ORS) was prepared from methyoxylamine hydrochloride (60 mg, 0.71 mmol), water (200 μL), pH 5.5 sodium acetate (200 μL) and 10 M sodium hydroxide (53.4 μL) and additional water (1.4 mL). A previously frozen sample of this buffer was further diluted with water by a factor of two to yield 1× ORS. Samples of 2′-deoxycytidine (˜0.5 mg) and 2′-deoxy-5-fluorocytidine (˜0.5 mg) were dissolved in 1 mL aliquots of the buffer. The samples were analysed by LC/MS immediately after making up the samples and at intervals while stored together at room temperature. Appearance of the reaction products 2′-deoxy-4(N)-methoxy cytidine (1) and 2′-deoxy-4(N)-methoxy-5-fluorocytidine (2) and a corresponding loss of starting material was observed. The corresponding loss of starting material relative to the reaction products is plotted on the graph below.

HPLC Method for LC/MS Analysis

| Column | Ascentis Express C18 | |
|---|---|---|
| | 15 × 4.6 mm, 5 μm | |
| Column temperature | 30° | C. |
| Flow rate | 1 | mL/min |
| Injection volume | 5 | uL |
| UV detection | 254 | nm |

| Solvent A | NH₄OAc pH 4.5 |
|---|---|
| Solvent B | Acetonitrile |
| Time (min) | % B |
| 0 | 1 |
| 1 | 1 |
| 10 | 50 |
| 12 | 50 |
| 13 | 1 |

Retention Times (mini

| | |
|---|---|
| 2′-Deoxycytidine | 2.38 |
| 2′-Deoxy-5-fluorocytidine | 3.20 |
| 2′-Deoxy-4(N)-methoxy cytidine (1) | 5.30 |
| 2′-Deoxy-4(N)-methoxy-5-fluorocytidine (2) | 5.58 |

Mass Spectra

2′-Deoxy-4(N)-methoxy cytidine m/z (ES⁺) 258 ([M+H, 100%); 280 ([M+Na], 50) and 537 ([2M+Na], 72). m/z (ES⁻) 256 ([M−H], 12%) and 316 ([M+AcOH-1], 100).

2′-Deoxy-4(N)-methoxy-5-fluorocytidine m/z (ES⁺) 276 ([M+H, 100%); 298 ([M+Na], 80) and 573 ([2M+Na], 20). m/z (ES⁻) 274 ([M−H], 100%) and 334 ([M+AcOH-1], 90%).

Proposed Structures of Reaction Products

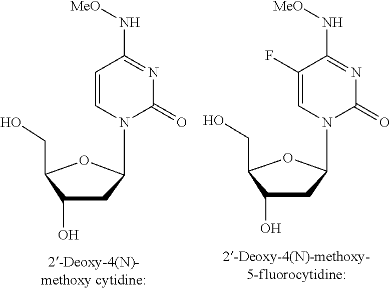

The data shows the 5-FdC is almost 10× more stable to methoxylamine treatment than canonical dC (see FIG. 9).

5-Fluoro C Experimental.

Scheme 1: Synthesis route for Fluoro C triphosphate. i. POCl₃, trimethyl phosphate, pyridine, dioxane; ii. (Bu₃N)₂.H₄P₂O₇, Bu₃N, acetonitrile; iii. Triethylammonium bicarbonate, H₂O, pH 7.6.

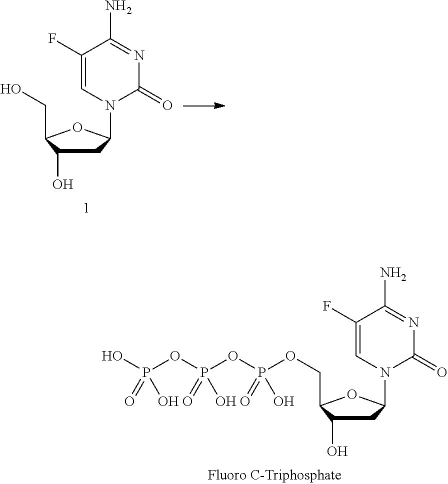

Scheme 2: Synthesis route for 3′-acetone oxime-2′-deoxy-5-fluorocytidine triphosphate (Fluoro C-oxime). (a) PPh₃, DIAD, MeCN; (b) KOH, H₂O; (c) TBDMSCI, imidazole, DMF; (d)N-hydroxy-4-nitrophthalimide, PPh₃, DIAD, THE; (e) i. MeNH₂, EtOH, ii. acetone; (f) (4-ClPhO)POCl₂ 3-nitro-1,2,4-triazole, pyridine; (g) 3HF.Et₃N, THE; (h) i. POCl₃, trimethyl phosphate, pyridine, dioxane; ii. (Bu₃N)₂.H₄P₂O₇, Bu₃N, acetonitrile; iii. Triethylammonium bicarbonate, H₂O, pH 7.6.

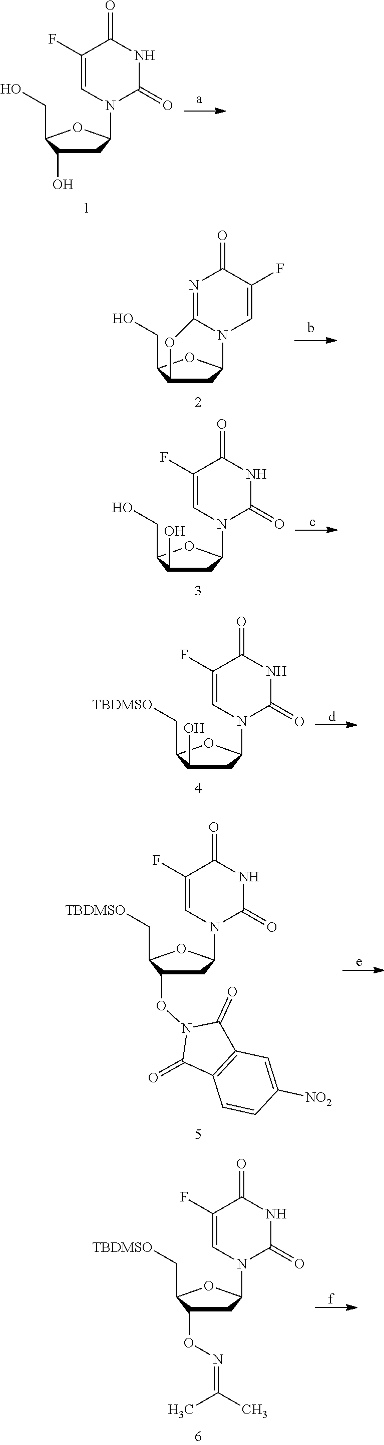

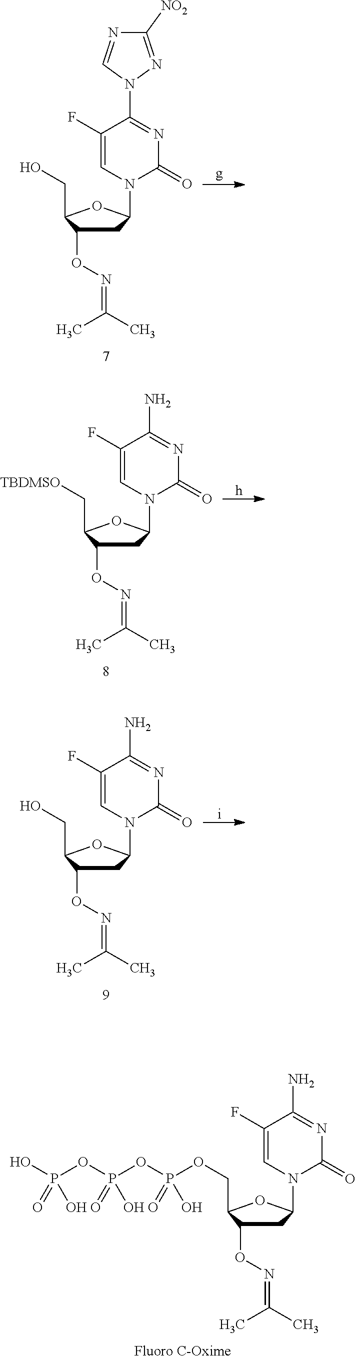

Scheme 3: Oxime deprotection of Fluoro C-oxime to yield Fluoro C-aminoxy. Prior to use, the oxime can be removed by incubation of the triphosphate Fluoro C-oxime in a solution of 1 M sodium acetate pH 5.5, 1.5% w/v methoxylamine, and ultrapure water for 60 minutes at room temperature.

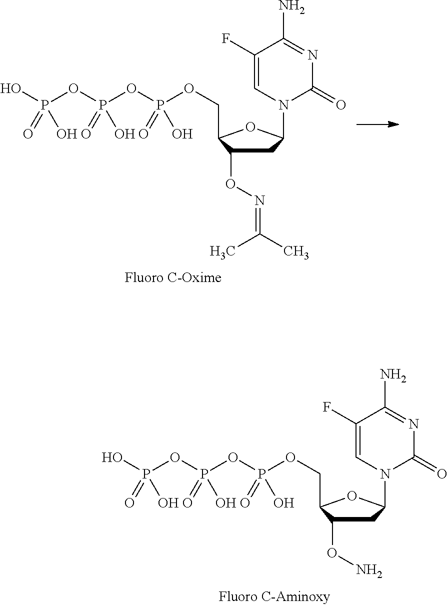

### 2′-Deoxy-5-Fluorocytidine 5′-Triphosphate

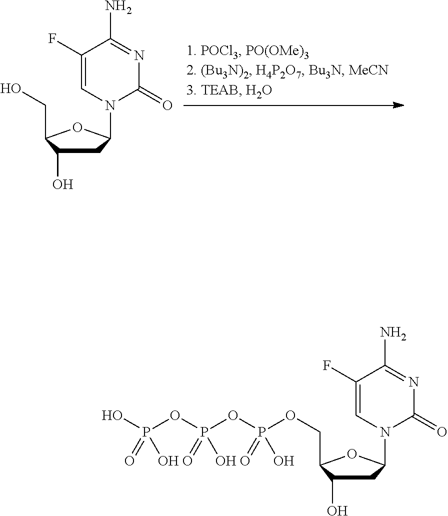

2′-Deoxy-5-fluorocytidine (250 mg, 1.02 mmol) was placed in a reaction flask, which was purged with nitrogen, then trimethyl phosphate (2.5 mL) was added. The suspension was cooled to 0° C. Phosphorus oxychloride (67 μl, 0.71 mmol) was added over 2 minutes, then the suspension was stirred for 8 minutes. Additional phosphorus oxychloride (67 μl, 0.71 mmol) was added over 2 minutes, then the solution was stirred at 0° C. for 35 minutes. Meanwhile, tributylammonium pyrophosphate (671 mg, 1.22 mmol) was suspended in anhydrous acetonitrile (3.8 mL) under nitrogen. Tributylamine (1.70 mL, 7.1 mmol) was added. The mixture was added to the reaction solution by syringe over 2 minutes while cooling in an ice/water bath. The mixture was stirred in an ice-water bath (0° C.) for 20 minutes, then 2 M pH 7.6 triethylammonium bicarbonate (2.5 mL) was added over 2 minutes and the mixture was allowed to warm to room temperature. Methyl-tert-butyl ether (7.5 mL) and water (5 mL) were added. The phases were separated and the aqueous phase was washed with methyl-tert-butyl ether (7.5 mL). The aqueous phase was concentrated using a rotary evaporator to ˜5 mL. Cold (warmed from −80° C. until the solid had just dissolved) 2% sodium perchlorate solution in acetone (30 mL) was added. The white emulsion was centrifuged for 20 minutes at 4000 rpm at −10° C. and the liquid was decanted. The white semi-solid was dissolved in water (2 mL) and cold 2% sodium perchlorate solution in acetone (30 mL) was added. The white emulsion was centrifuged for 20 minutes at 4000 rpm at −10° C. and the liquid was decanted. The sodium perchlorate precipitation was repeated and the solid was washed with cold acetone (2×1 mL) to give crude triphosphate sodium salt as a white solid (816 mg). This was dissolved in water to 2.3 mL and 165 mL of the solution (approximately 7% of the crude) was purified by reverse phase HPLC using a Supelco Ascentis C18 column (25 cm×10 mm, 5 μm), flow rate 3 mL/min, and a gradient of A: 100 mM triethylammonium bicarbonate pH 7.5, B: acetonitrile; A to 30% B over 32 minutes (6 runs). After evaporation of solvent from product containing fractions, methanol (5 mL) was added to each and the solvent was evaporated. The residue was dissolved in water (4 mL), the solution was frozen and lyophilised to give semi-purified triphosphate triethylamine salt as colourless granular crystals (14 mg). This was dissolved in water (450 microlitres) and purified by ion-exchange chromatography using a Source15Q column (10×150 mm), flow rate 3 mL/min, and gradient of 10 mM triethylammonium bicarbonate pH 7.5 to 1 M triethylammonium bicarbonate pH 7.5 over 22 minutes, followed by hold for 10 minutes (4 runs). After evaporation of solvent from product-containing fractions, methanol (5 mL) was added and the solvent was evaporated. The residue was dissolved in water (4 mL) and the solution was frozen and lyophilised to give 2′-deoxy-5-fluorocytidine 5′-triphosphate tris(triethylamine) salt as colourless granular crystals (11.5 mg, 1.4% from purification of ˜7% of available crude); m/z (ES⁻) 484 ([M−H], 100%); ¹H NMR (400 MHz, D₂O) d (ppm) 8.02 (1H, d, J=6.4 Hz), 6.21 (1H, td, J=6.5, 3.3 Hz), 4.58 (1H, dt, J=6.5, 3.3 Hz), 4.18 (2H, m), 4.14 (1H, m), 3.14 (18H, q, J=7.3 Hz), 2.37 (1H, ddd, J=14.0, 6.2, 4.1 Hz), 2.25 (1H, dt, J=13.8, 6.8 Hz) and 1.22 (27H, t, J=7.3 Hz); ¹⁹F NMR (376 MHz, D₂O) d (ppm) −164.44; ³¹P NMR (162 MHz, D₂O) d (ppm) −9.64 (d, J=19.9 Hz), −11.6 (d, J=19.9 Hz) and −23.12 (d, J=20.0 Hz).

### 2,3′-Cyclo-2′-Deoxy-5-Fluorouridine

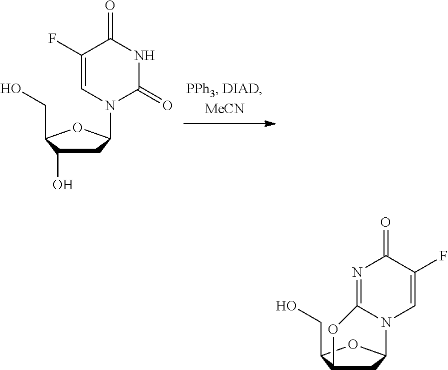

2′-Deoxy-5-fluorouridine (1.00 g, 4.06 mmol) and triphenylphosphine (2.13 g, 8.12 mmol) were placed in a reaction flask. This was purged with nitrogen, then anhydrous acetonitrile (10 mL) was added. The suspension was cooled to −5° C. then diisopropyl azobisdicarboxylate (1.60 mL, 8.12 mmol) was added over 15 minutes while stirring vigorously. The pale yellow suspension was stirred for a further 15 minutes at −2° C. The suspension was allowed to warm to room temperature (20° C.) over 5 minutes, then the pale yellow solution was stirred for a further 30 minutes. Ethyl acetate (10 mL) was added, and the solution was cooled in an ice-water bath. After 30 minutes, more ethyl acetate (10 mL) was added, and the suspension was cooled again in an ice-water bath. The sticky solid which formed was non-filterable, so the solvent was removed using a rotary evaporator. Ethyl acetate (10 mL) was added, the suspension was stirred at room temperature for 15 minutes, then filtered and the solid was washed with ethyl acetate (3×1 mL), and dried to give an approximately 1.8:1.8:1 molar mixture of triphenylphosphine oxide, diisopropyl hydrazinedicarboxylate and 2,3′-cyclo-2′-deoxy-5-fluorouridine as a pale yellow solid (757 mg). The filtrate was allowed to stand at room temperature for 16 h, stirred for 15 minutes, then filtered and the solid was washed with ethyl acetate (3×1 mL) to give additional approximately 1.8:1.8:1 molar mixture of triphenylphosphine oxide, diisopropyl hydrazinedicarboxylate and 2,3′-cyclo-2′-deoxy-5-fluorouridine as a white solid (471 mg, total 1.23 g, containing approximately 204 mg (22% isolated yield) of 2,3′-cyclo-2′-deoxy fluorouridine, the remainder being triphenylphosphine and diisopropyl hydrazinedicarboxylate; m/z (ES⁺) 229 ([M+H], 100%), 251 ([M+Na], 7), 457 ([2M+H], 68), 479 ([2M+Na], 100) and 495 ([2M+K], 14%); ¹H NMR (400 MHz, DMSO-d6) δ (ppm) 8.12 (d, J=5.3 Hz), 5.83 (d, J=3.7 Hz), 5.31 (1H, m), 5.07 (1H, t, J=5.4 Hz), 4.22 (1H, td, J=6.3, 2.3 Hz), 2.63 (1H, d, J=12.9 Hz) and 2.48 (1H, m).

xylo-2′-Deoxy-5-Fluorouridine

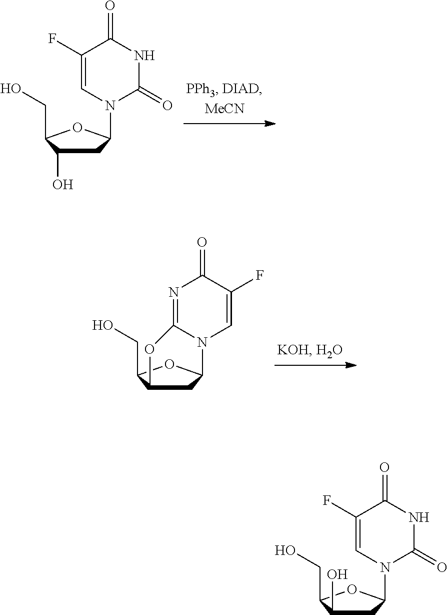

2′-Deoxy-5-fluorouridine (5.0 g, 20.3 mmol) and triphenylphosphine (7.46 g, 28.4 mmol) were placed in a flask. This was purged with nitrogen, then anhydrous acetonitrile (51 mL) was added. The suspension was cooled to −5 to −10° C., then diisopropyl azobisdicarboxylate (5.6 mL, 28.4 mmol) was added over 15 minutes while stirring vigorously. The pale yellow suspension was stirred for a further 15 minutes at −5 to −2° C., allowed to warm to room temperature over 10 minutes, then the solution was stirred for a further 30 minutes. Most of the solvent was removed using a rotary evaporator, then water (50 mL) was added. The suspension was stirred at room temperature for 1 h, filtered, and the solid residue of triphenylphosphine oxide:diisopropyl hydrazinedicarboxylate complex was washed with water (4×20 mL). The filtrate was extracted with dichloromethane (25 mL), then the aqueous phase containing 2,3′-cyclo-2′-deoxy-5-fluorouridine was concentrated using a rotary evaporator to ˜50 mL. Potassium hydroxide (1.75 g, 31.2 mmol) was added. The pale yellow solution was stirred at room temperature for 1 h, then neutralised with freshly washed Dowex 50WX8 acidic resin (8.0 g) to pH 7.6. The resin was removed by filtration, then the solution (still cloudy) was filtered through Celite®. The solvent was evaporated using a rotary evaporator to give a white solid. Isopropanol (50 mL) was added, and the solvent was removed using a rotary evaporator. Toluene (50 mL) was added. The solvent was removed using a rotary evaporator. This was repeated to give crude xylo-2′-deoxy-5-fluorouridine as a pale yellow solid (5.12 g); m/z (ES⁺) 247 ([M+H], 40%), 269 ([M+Na], 100) and 285 ([M+K], 12%); m/z (ES⁻) 245 ([M−H], 100%) and 491 ([2M−H], 84); ¹H NMR (400 MHz, DMSO-d6) d (ppm) 11.8 (1H, br s), 7.86 (1H, d, J=7.5 Hz), 6.03 (1H, dt, J=8.5, 2.3 Hz), 5.5 (1H, br s), 4.8 (1H, br s), 4.19 (1H, dd, J=5.0, 2.9 Hz), 3.69 (1H, dd, J=12.8, 5.0, Hz), 3.58 (1H, dd, J=12.8, 7.9 Hz), 2.48 (m) and 1.79 (1H, dd, J=14.5, 2.5 Hz); ¹⁹F NMR (376 MHz, DMSO-d6) d (ppm) −166.13.

### 5′(O)-tert-Butyldimethylsilyl-xylo-2′-Deoxy-5-Fluorouridine

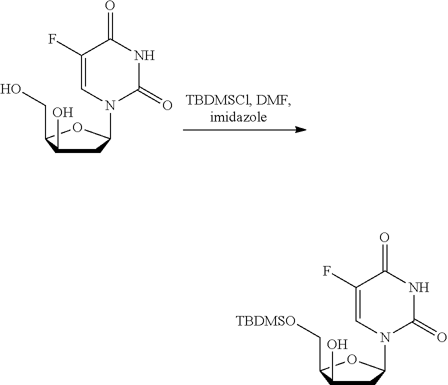

Crude xylo-2′-deoxy-5-fluorouridine (5.12 g) and imidazole (2.83 g, 41.6 mmol) were placed in a flask. This was purged with nitrogen and anhydrous DMF (48 mL) was added. The suspension was cooled in an ice bath to 0-5° C. A solution of tert-butyldimethylchlorosilane (3.76 g, 25.0 mmol) in anhydrous DMF (8.0 mL) was added over 30 minutes while cooling in an ice bath, maintaining the temperature at 5-7° C. The solution was stirred at 0-5° C. for 30 minutes, allowed to warm to room temperature, then allowed to stir for 21 h, when a solution of additional tert-butyldimethylchlorosilane (470 mg, 3.12 mmol) in anhydrous DMF (1.2 mL) was added. The solution was stirred at room temperature for 2 h, then cooled in an ice-water bath to ˜15° C. and quenched with methanol (2.0 mL, 50 mmoL). The suspension was stirred at room temperature for 30 minutes, then cooled in an ice-water bath to 10-15° C., and water (280 mL) was added over 30 minutes in portions. After the addition of the first 60 mL, an oily liquid started to separate. At this point, the mixture was seeded with a small quantity of product. The suspension was stirred at room temperature for 18 h, then filtered. The solid precipitate was washed with water (4×10 mL) and dried to give 5′(0)-tert-butyldimethylsilyl-xylo-2′-deoxy-5-fluorouridine as a white solid (3.89 g, 53% over 3 steps from 2′-deoxy-5-fluorouridine); m/z (ES⁺) 361 ([M+H], 100) and 383 ([M+H], 68); m/z (ES⁻) 359 ([M−H], 100) and 719 ([2M+H], 8); ¹H NMR (400 MHz, CD₃CN) d (ppm) 9.22 (1H, br s), 8.10 (1H, d, J=7.4 Hz), 6.09 (1H, dt, J=8.3, 2.0 Hz), 4.35 (1H, m), 4.00 (1H, dd, J=11.1, 4.9 Hz), 3.93 (1H, dd, J=11.1, 5.6 Hz), 3.86 (1H, ddd, J=5.4, 5.0, 3.1 Hz), 3.61 (1H, d, J=3.3 Hz), 2.56 (1H, J=14.6, 8.4, 5.3 Hz), 1.95 (1H, dd, J=14.5, 2.2, 0.9 Hz), 0.89 (9H, s), 0.08 (3H, s) and 0.08 (3H, s); ¹⁹F NMR (376 MHz, CD₃CN) d (ppm) −168.24.

### 3′-O-(4-Nitrophthalimido)-5′(O)-tert-Butyldimethylsilyl-2′-Deoxy-5-Fluorouridine

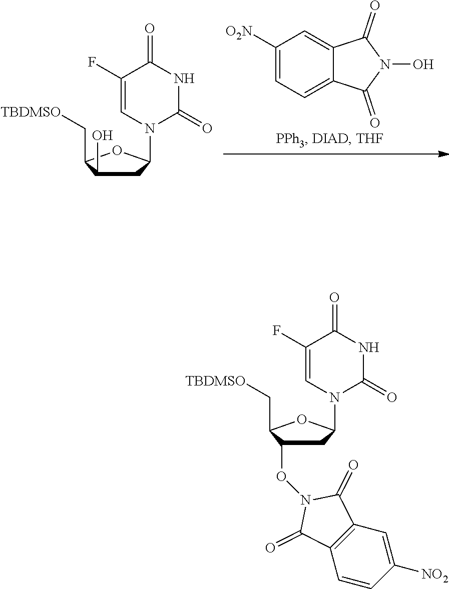

5′(0)-tert-Butyldimethylsilyl-xylo-2′-deoxy-5-fluorouridine (100 mg, 0.28 mmol), triphenylphosphine (0.182 g, 0.69 mmol) and N-hydroxy-4-nitrophthalimide (0.144 mg, 0.69 mmol) were placed in a reaction flask. This was purged with nitrogen, then anhydrous THF (1.8 mL) was added. The solution was cooled in an ice-water bath, then diisopropyl azobisdicarboxylate (0.14 mL, 0.70 mmol) was added over 20 minutes. The deep brown solution was stirred in an ice-water bath for 30 minutes, then allowed to warm to room temperature and stirred at room temperature for 30 minutes. Toluene (5 mL) was added, and the solution was washed with saturated sodium bicarbonate solution (4×5 mL). The organic layer was dried (MgSO₄), filtered and the solvent was evaporated using a rotary evaporator. The product was purified by flash chromatography using a pre-packed silica cartridge (12 g) with a dichloromethane to dichloromethane-ethyl acetate (75:25) gradient, followed by a second purification using a pre-packed silica cartridge (12 g) with a dichloromethane to dichloromethane-ethyl acetate (90:10) gradient to give 3′-O-(4-nitrophthalimido)-5′(O)-tert-butyldimethylsilyl-2′-deoxy-5-fluorouridine as a white solid (90 mg, 50%); m/z (ES⁺) 551 ([M+H], 100%) and 568 ([M+Na], 17); m/z (ES⁻) 549 ([M−H], 100%); ¹H NMR (400 MHz, CD₃CN) d (ppm) 9.27 (1H, br s), 8.62 (1H, dd, J=8.2, 2.0 Hz), 8.57 (1H, dd, J=2.0, 0.4 Hz), 8.06 (1H, dd, J=8.1, 0.4 Hz), 7.90 (1H, d, J=6.7 Hz), 6.36 (1H, td, J=7.1, 1.7 Hz), 4.97 (1H, dt, J=5.4, 1.4 Hz), 4.40 (1H, m), 3.90 (2H, m), 2.66 (1H, dd, J=14.8, 5.6 Hz), 2.14 (1H, ddd, J=14.8, 8.6, 5.5 Hz), 0.88 (9H, s), 0.10 (3H, s) and 0.08 (3H, s); ¹⁹F NMR (376 MHz, CD₃CN) d (ppm) −167.93.

### 3′-O—(N-Acetone Oxime)-5′(O)-tert-Butyldimethylsilyl-xylo-2′-Deoxy-5-Fluorouridine

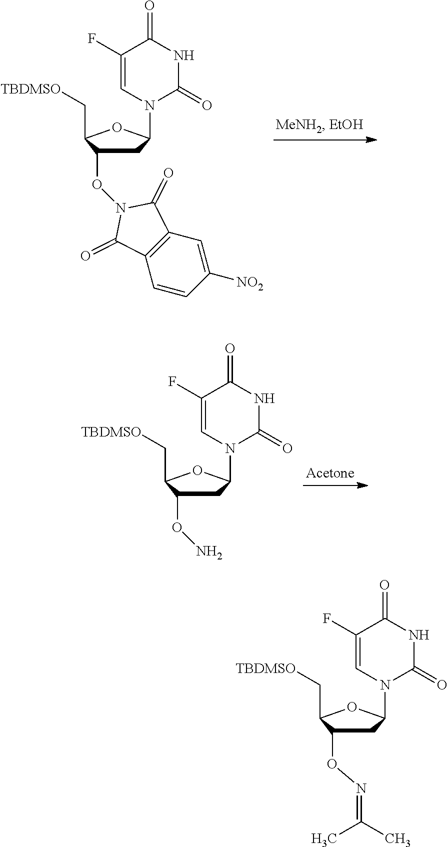

Methylamine (33 wt % in ethanol, 1.55 mL, 13.1 mmol) was added to 3′-O-(4-nitrophthalimido)-5′(O)-tert-butyldimethylsilyl-2′-deoxy-5-fluorouridine (90 mg, 0.16 mmol). The solution was stirred for 3 h at room temperature, then cooled in an ice-water bath and acetone (1.50 mL, 20.4 mmol) was added over 1 minute. The solution was allowed to warm to room temperature and stirred for 1 h. Toluene (5 mL) and 1 M citric acid (5 mL) were added. The mixture was shaken, the layers allowed to separate, and the organic layer was washed with 1 M citric acid (5 mL) and brine (4×5 mL), dried (MgSO₄) and filtered. The solvent was removed using a rotary evaporator to give 3′-O—(N-acetone oxime)-5′(0)-tert-butyldimethylsilyl-2′-deoxy-5-fluorouridine as a pale brown solid (67 mg, 98%); m/z (ES⁺) 286 ([M+H-5-fluorouracil], 40%), 416 ([M+H], 100) and 438 ([M+Na], 32); m/z (ES⁻) 414 ([M−H], 100); ¹H NMR (400 MHz, CD₃CN) d (ppm) 9.25 (1H, br s), 7.99 (1H, d, J=6.9 Hz), 6.16 (td, J=6.9, 1.9 Hz), 4.68 (dt, J=6.1, 1.8 Hz), 4.15 (q, J=2.3 Hz), 3.93 (1H, dd, J=11.5, 2.6 Hz), 3.83 (1H, dd, J=11.5, 2.5 Hz), 2.44 (1H, ddd, J=14.0, 5.9, 1.9 Hz), 2.09 (1H, ddd, J=14.1, 7.9, 6.2 Hz), 1.829 (3H, s), 1.827 (3H, s), 0.91 (9H, s), 0.12 (3H, s) and 0.11 (3H, s); ¹⁹F NMR (376 MHz, CD₃CN) d (ppm) −168.24.

### 3′-O—(N-Acetone Oxime)-5′(O)-tert-Butyldimethylsilyl-2′-Deoxy-5-Fluorocytidine

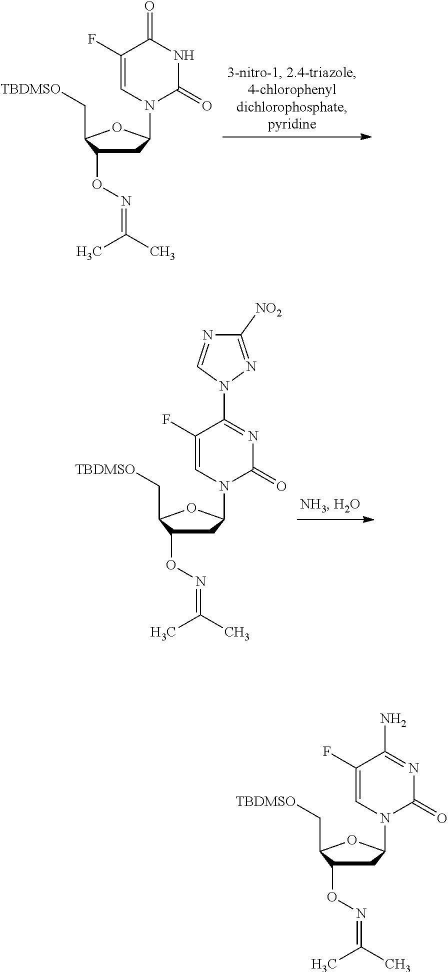

3′-O—(N-Acetone oxime)-5′(0)-tert-butyldimethylsilyl-2′-deoxy-5-fluorouridine (716 mg, 1.72 mmol) and 3-nitro-1,2,4-triazole (590 mg, 5.17 mmol) were placed in a reaction flask. This was purged with nitrogen, anhydrous pyridine (3.2 mL) was added, then the solution was cooled in an ice-water bath. 4-Chlorophenyl dichlorophosphate (0.42 mL, 2.58 mmol) was added over 5 minutes. The pale brown suspension was stirred in an ice-water bath for 1 h, then allowed to warm to room temperature. THF (3.2 mL) was added, then the suspension was cooled in an ice-water bath. Aqueous ammonia (SG 0.88, 1.9 mL, ˜34 mmol) was added over 5 minutes while cooling. The mixture was allowed to warm to room temperature, then stirred at room temperature for 30 minutes. Toluene (25 mL), 1 molar citric acid (15 mL) and ethyl acetate (10 mL) were added. The phases were separated and the organic phase was washed with 1 M citric acid (35 mL), saturated sodium bicarbonate (35 mL), dried (MgSO₄) and filtered, then re-filtered through Celite®. The solvent was removed using a rotary evaporator, and the product was purified by flash chromatography using a pre-packed silica cartridge (80 g) in three approximately equal portions using a dichloromethane to dichloromethane-methanol (95:5) gradient to give 3′-O—(N-acetone oxime)-5′(0)-tert-butyldimethylsilyl-2′-deoxy-5-fluorocytidine as a white solid (653 mg, 91%); m/z (ES⁺) 286 ([M+H-5-fluorocytosine], 74%), 829 ([2M+H], 100) and 851 ([2M+Na], 25); m/z (ES⁻) 413 ([M−H], 100%); ¹H NMR (400 MHz, CD₃CN) d (ppm) 7.97 (1H, d, J=7.0 Hz), 6.13 (dt, J=5.9, 1.9 Hz), 6.12 (2H, br s), 4.67 (dt, J=6.1, 2.0 Hz), 4.15 (1H, q, J=2.4 Hz), 3.92 (1H, dd, J=11.4, 2.7 Hz), 3.82 (1H, dd, J=11.4, 2.7 Hz), 2.49 (1H, ddd, J=13.9, 5.9, 2.1 Hz), 2.02 (1H, ddd, J=14.0, 7.8, 6.2 Hz), 1.83 (3H, s), 1.82 (3H, s), 0.90 (9H, s), 0.11 (3H, s) and 0.10 (3H, s); ¹⁹F NMR (376 MHz, CD₃CN) d (ppm) −169.70 (4-NH₂ isotopomer) and 169.77 (4-NHD isotopomer).

### 3′-O—(N-Acetone Oxime)-2′-Deoxy-5-Fluorocytidine

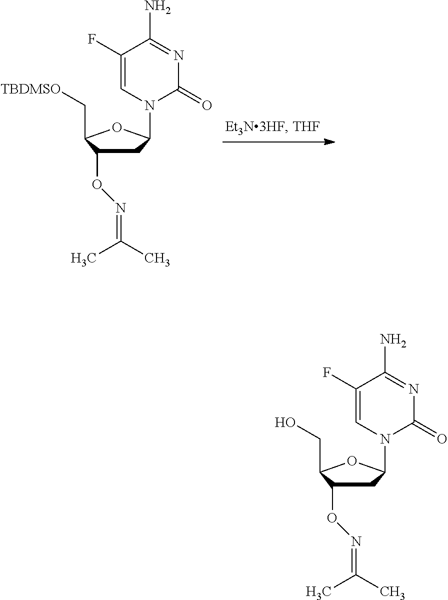

3′-O—(N-Acetone oxime)-5′(0)-tert-butyldimethylsilyl-2′-deoxy-5-fluorocytidine (653 mg, 1.57 mmol) was suspended in anhydrous THF (5.4 mL). Triethylamine trihydrofluoride (0.77 mL, 4.7 mmol) was added over 2 minutes, then the solution was stirred at room temperature for 20 h. Ethoxytrimethylsilane (2.5 mL, 16 mmol) was added over 10 minutes, the solution was stirred at room temperature for 1 h, then the solvent was removed using a rotary evaporator. THF (5 mL) was added and the solvent was evaporated to give a white foam. Methyl-tert-butyl ether (10 mL) was added. A white solid formed. The suspension was stirred at room temperature for 1 h, then filtered and the solid was washed with methyl-tert-butyl ether (3×5 mL) and dried to give 3′-O—(N-acetone oxime)-2′-deoxy-5-fluorocytidine as a fine white solid (426 mg, 90%); m/z (ES⁺) 172 ([M+H-5-fluorocytosine], 48%), 601 ([2M+H], 100), 623 ([2M+Na], 58); m/z (ES⁻) 299 ([M−H), 100%); ¹H NMR (400 MHz, CD₃CN) d (ppm) 7.95 (1H, d, J=7.0 Hz), 6.3 (1H, br s), 6.1 (1H, br s), 6.09 (1H, td, J=6.9, 1.4 Hz), 4.68 (1H, dt, J=6.2, 2.0 Hz), 4.10 (1H, q, J=2.3 Hz), 3.74 (1H, ddd, J=11.8, 4.5, 3.4 Hz), 3.68 (1H, ddd, J=11.9, 5.2, 3.4 Hz), 3.45 (1H, t, J=5.1 Hz), 2.43 (1H, ddd, J=13.9, 5.8, 1.7 Hz), 2.12 (1H, ddd, J=14.0, 7.7, 6.4 Hz), 1.832 (3H, s) and 1.827 (3H, s); ¹⁹F NMR (376 MHz, CD₃CN) d (ppm) −169.74 (4-NH₂ isotopomer) and 169.81 (4-NHD isotopomer).

### 3′-O—(N-Acetone Oxime)-2′-Deoxy-5-Fluorocytidine-5′-Triphosphate

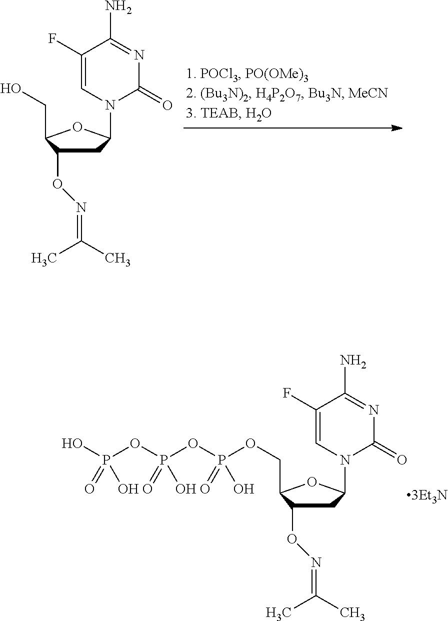

3′-O—(N-Acetone oxime)-2′-deoxy-5-fluorocytidine (100 mg, 0.33 mmol) was dried by co-evaporation with toluene (2×2 mL). The reaction flask was purged with nitrogen, then trimethyl phosphate (1.0 mL) was added. The suspension was cooled to 0° C. in an ice-water bath. Phosphorus oxychloride (22 μL, 0.23 mmol) was added over 2 minutes, then the solution was stirred for 8 minutes. Additional phosphorus oxychloride (22 μL, 0.23 mmol) was added over 2 minutes, then the solution was stirred at 0° C. for 35 minutes. Meanwhile, tributylammonium pyrophosphate (219 mg, 0.400 mmol) was suspended in anhydrous acetonitrile (1.5 mL) under nitrogen. Tributylamine (0.56 mL, 2.33 mmol) was added. The mixture was added to the reaction solution by syringe while stirring vigorously over 2 minutes while cooling in a water/ice bath, then the solution was stirred in an ice-water bath (0° C.) for 20 minutes. 2 M pH 7.6 Triethylammonium bicarbonate (1 mL) was added over 2 minutes, then the mixture was allowed to warm to room temperature. Water (5 mL) and methyl-tert-butyl ether (5 mL) were added and the layers were separated. The organic layer was extracted with water (1 mL) and the combined aqueous phases were concentrated using a rotary evaporator (bath temperature 30° C.). Cold 2% sodium perchlorate (−70° C.) solution in acetone (15 mL) was added to the residue, then the white suspension was centrifuged for 20 minutes at 4000 rpm at −10° C., and the liquid was decanted. The solid was dissolved in water (1 mL) and cold 2% sodium perchlorate (−70° C.) solution in acetone (15 mL) was added. The white suspension was centrifuged for 20 minutes at 4000 rpm at −10° C., the liquid was decanted and the solid was washed with cold acetone (2×1 mL) and air-dried to give crude triphosphate sodium salt as a white solid (416 mg). This was dissolved in water (2 mL) and purified by reverse phase HPLC using a using a Phenomenex Kinetex C18 column (30×250 mm, 5 μm), flow rate 25 mL/min, and A: 100 mM triethylammonium bicarbonate pH 7.5, B: Acetonitrile; 2% B for 2 minutes then a gradient to 25% B over 22 minutes then 25% B for 5 minutes (4 runs). The product-containing fractions were combined and the solvent was removed using a rotary evaporator. Methanol (10 mL) was added, and the solvent was evaporated using a rotary evaporator. This was repeated. Water (15 mL) was added and most of the solvent was removed using a rotary evaporator. Water (5 mL) was added and the solution was frozen then lyophilised to give semi-purified triphosphate triethylamine salt as a white glassy solid (138 mg). This was dissolved in water (2 mL) and purified by ion-exchange chromatography using a Source15Q column (50×200 mm), flow rate 35 mL/min, and gradient of 10 mM triethylammonium bicarbonate pH 7.5 to 1 M triethylammonium bicarbonate pH 7.5 over 32 minutes, followed by hold for 5 minutes (2 runs).

After evaporation of solvent from product containing fractions, methanol (10 mL) was added, then the solvent was removed using a rotary evaporator. This was repeated. Water (15 mL) was added, then the solution was concentrated to ˜5 mL using a rotary evaporator. The sample was frozen, then lyophilised to give 3′-O—(N-acetone oxime)-2′-deoxy-5-fluorocytidine-5′-triphosphate triethylamine salt as white glassy solid (84 mg, 24%); m/z (ES⁻) 539 ([M−H], 100%); ¹H NMR (400 MHz, D₂O) d (ppm) 8.06 (1H, d, J=6.3 Hz), 6.16 (1H, ddd, J=9.0, 5.9, 1.5 Hz), 4.90 (1H, d, J=5.4 Hz), 4.38 (1H, m), 4.18 (2H, m), 3.14 (18H, J=7.3 Hz), 2.53 (1H, dd, J=14.3, 5.6 Hz), 2.23 (1H, ddd, J=14.4, 8.9, 5.6 Hz), 1.89 (3H, s), 1.87 (3H, s) and 1.22 (27H, t, J=7.3 Hz; ¹⁹F NMR (376 MHz, D₂O) d (ppm) −164.04; ³¹P NMR (162 MHz, D₂O) d (ppm) −9.87 (d, J=20.0 Hz), −11.66 (d, J=19.8 Hz) and −23.25 (t, J=20.0 Hz).

### DESCRIPTION OF FIGURES

FIG. 1: Stability of 5-F vs. Canonical 2′-Deoxycytidine in 700 mM pH 5.5 Nitrite at room temperature.

FIG. 2: Stability of 5-Me vs. Canonical 2′-Deoxycytidine in 700 mM pH 5.5 Nitrite at room temperature.

FIG. 3: Stability of 2′-Deoxycytidines in 700 mM pH 5.5 Nitrite at room temperature.

FIG. 4: Stability of 2′-Deoxycytidines in 700 mM pH 5.5 Nitrite at room temperature.

FIG. 5: Deamination products of 2-Deoxy-5-propynylcytidine in 700 mM pH 5.5 Nitrite at room temperature.

FIG. 6: Deamination products of 2-Deoxy-5-propynylcytidine in 700 mM pH 5.5 Nitrite.

FIG. 7: Stability of 2′-Deoxycytidines in 700 mM pH 5.5 Nitrite at room temperature.

FIG. 8: Stability of 5-substituted 2′-Deoxycytidines in 700 mM pH 5.5 Nitrite at room temperature.

FIG. 9: Stability of 2′-Deoxycytidines in 1× ORS at room temperature.

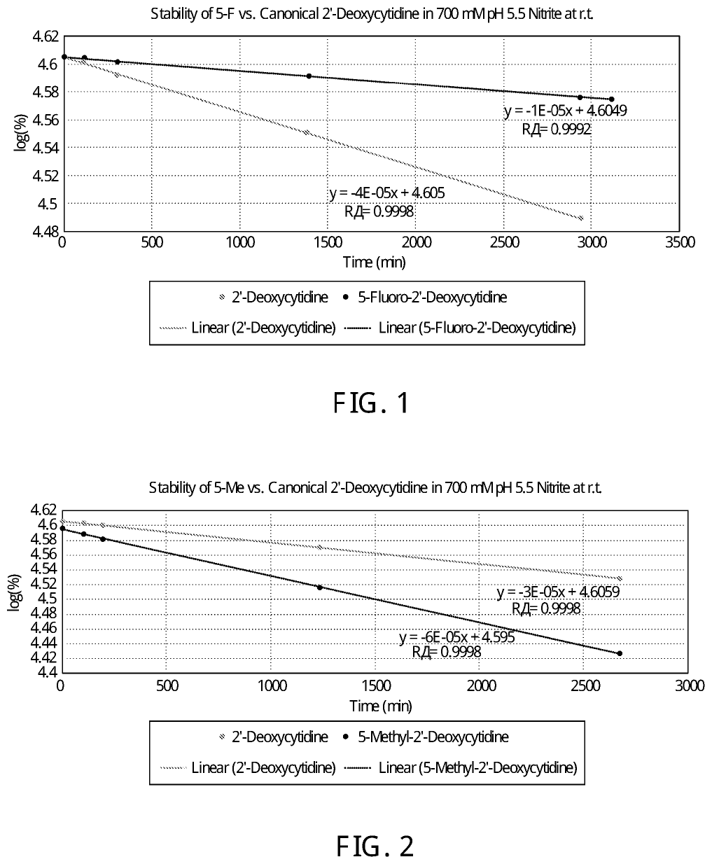

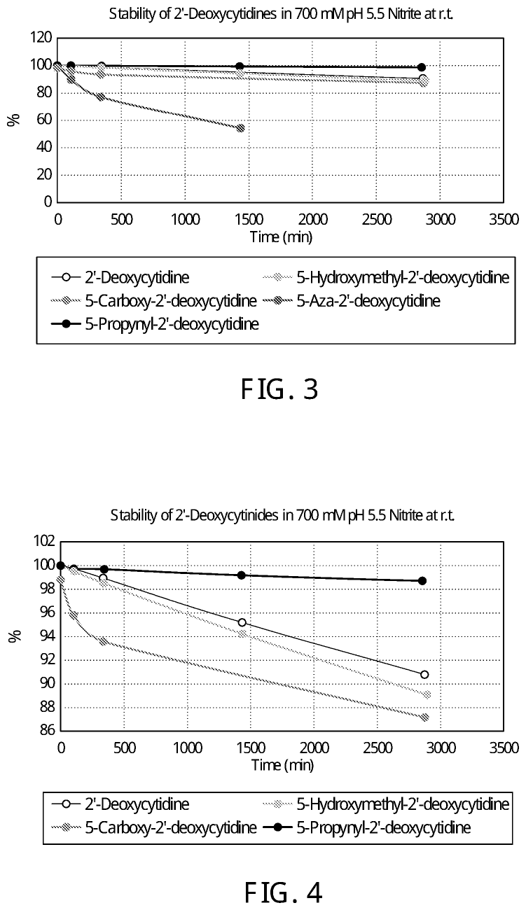

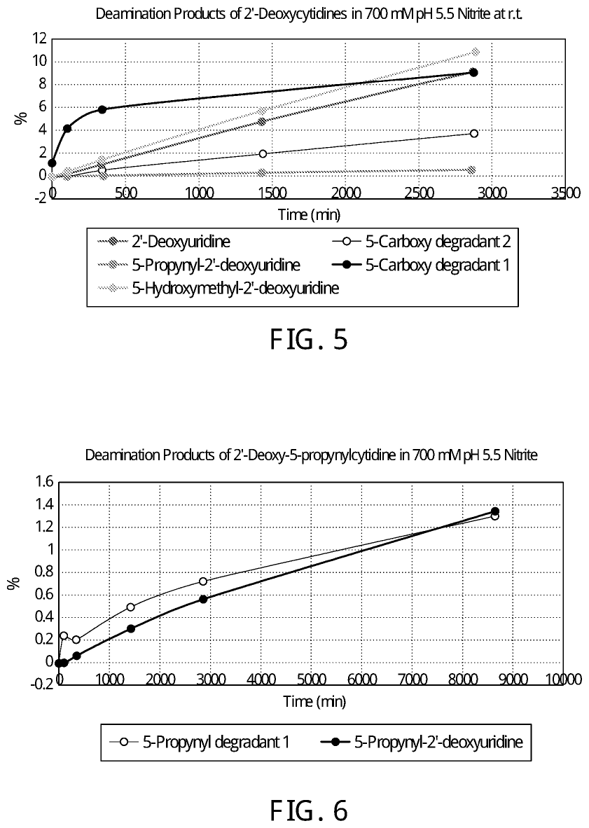

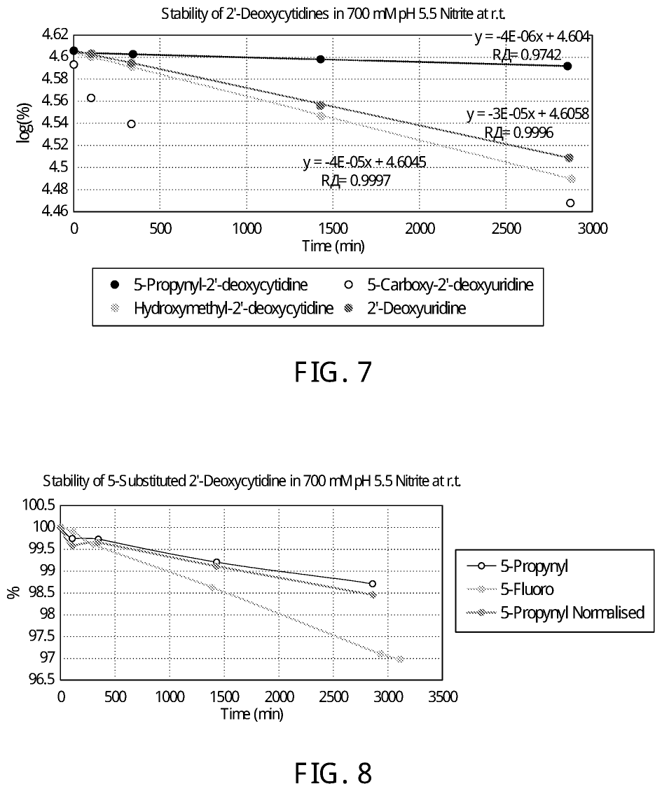

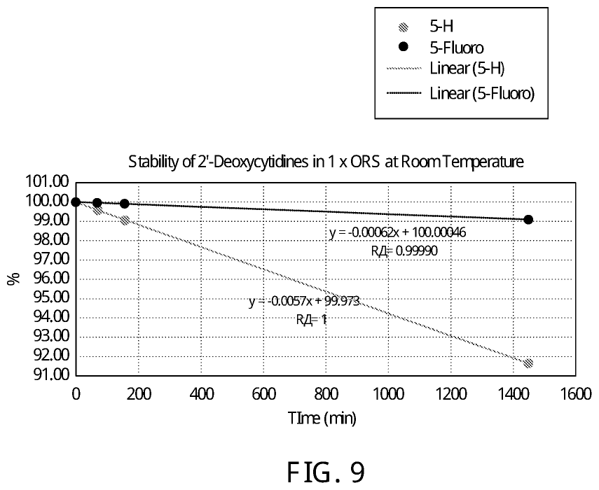

1. A compound according to Formula (1c) or (1d): 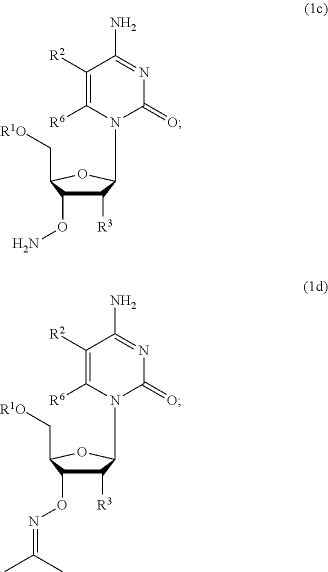
wherein,
R¹ is a phosphate or polyphosphate group or salt thereof, optionally containing one or more sulfur atoms;
R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe;
wherein R⁴ and R⁵ are independently selected from H and C₁₋₆ alkyl optionally substituted with OH or halo atoms; and
R⁶ is H or D.

2. The compound according to claim 1 according to Formula (1a) or (1b): 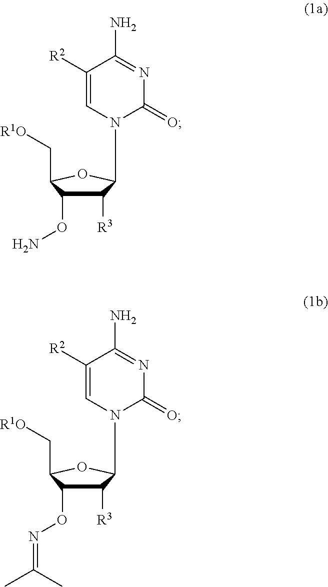
wherein,
R¹ is a phosphate or polyphosphate group or salt thereof, optionally containing one or more sulfur atoms;
R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and
R³ is selected from H, OH, F, OCH₃, or OCH₂CH₂OMe;
wherein R⁴ and R⁵ are independently selected from H and C₁₋₆ alkyl optionally substituted with OH or halo atoms.

3. The compound according to claim 1 or 2, wherein R¹ is —(PO₃)⁻ₓ(PO₂S)⁻_{y}(PO₃)⁻, where x, y and z are independently 0-5 and x+y+z is 1-5.

4. The compound according to claim 3, wherein R¹ is a monophosphate, diphosphate, triphosphate, tetraphosphate, pentaphosphate, or (alpha-thio)triphosphate group.

5. The compound according to any one of claims 1 to 4, wherein R² is selected from the group consisting of: fluoro; propynyl and but-3-yn-1-ol.

6. The compound according to any one of claims 1 to 5, wherein R³ is H.

7. The compound according to any one of claims 3 to 6, wherein R⁶ is D.

8. The compound according to claim 1, which is selected from the group consisting of: 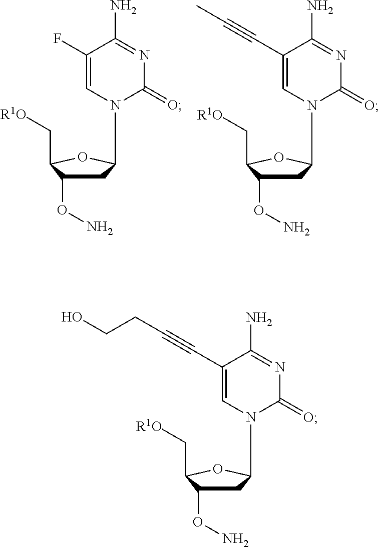
wherein R¹ is a phosphate or polyphosphate group or salt thereof, optionally containing one or more sulfur atoms.

9. The compound according to any one of claims 1 to 8, wherein R¹ is a triphosphate or (alpha-thio)triphosphate group.

10. The compound according to claim 1, which is selected from the group consisting of: 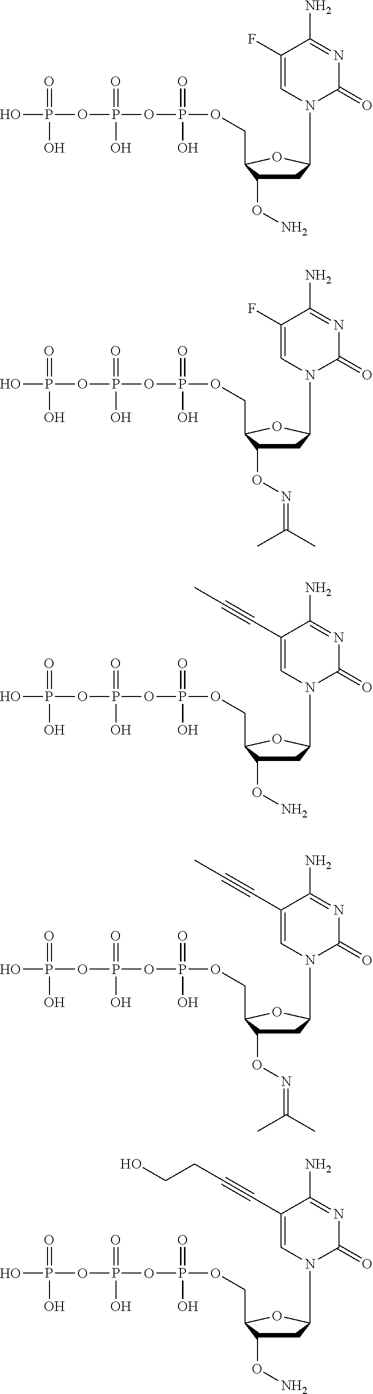 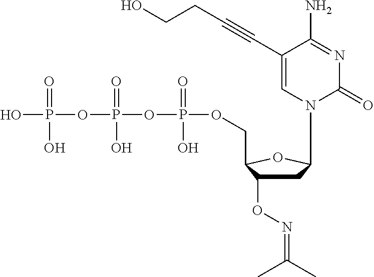
or a salt thereof.

11. A method of nucleic acid synthesis comprising reacting a compound according to any one of claims 1 to 10 with an oligonucleotide in the presence of a polymerase or terminal deoxynucleotidyl transferase (TdT) enzyme and treating the extended oligonucleotide with a nitrite salt.

12. The method according to claim 11, wherein the oligonucleotide sequence is a solid-supported oligonucleotide sequence.

13. The method according to claim 11 or 12, wherein the nitrite salt is sodium nitrite.

14. A method of synthesizing a compound according to formula (1a): 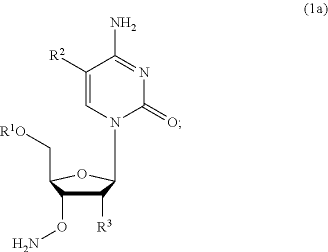
wherein,
R¹ is a phosphate or polyphosphate group or salt thereof, optionally containing one or more sulfur atoms;
R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and
R³ is selected from H, OH, F, OCH₃, or OCH₂CH₂OMe;
wherein R⁴ and R⁵ are independently selected from H and C₁₋₆ alkyl optionally substituted with OH or halo atoms comprising taking a compound according to Formula (1b): 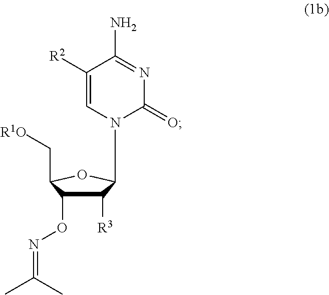
wherein,
R¹ is a phosphate or polyphosphate group or salt thereof, optionally containing one or more sulfur atoms;
R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and
R³ is selected from H, OH, F, OCH₃, or OCH₂CH₂OMe;
wherein R⁴ and R⁵ are independently selected from H and C₁₋₆ alkyl optionally substituted with OH or halo atoms
and treating the compounds of Formula (1b) with an aminooxy compound.

15. The method according to claim 14, wherein the aminooxy compound is hydroxylamine, methoxylamine or ethoxylamine.

16. A kit comprising:
(i) a compound according to any one of claims 1 to 10;
(ii) a polymerase or terminal deoxynucleotidyl transferase (TdT) enzyme; and optionally
(iii) a nitrite salt.

17. An oligonucleotide according to Formula (2c) or (2d): 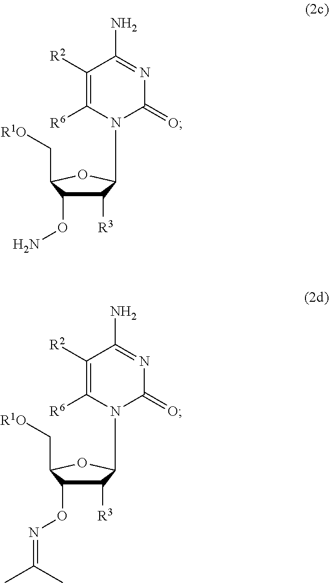
wherein,
R¹ is an oligonucleotide;
R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵;
R³ is selected from H, OH, F, OCH₃, or OCH₂CH₂OMe;
wherein R⁴ and R⁵ are independently selected from H and C₁₋₆ alkyl optionally substituted with OH or halo atoms; and
R⁶ is H or D.

18. The oligonucleotide according to claim 17 according to Formula (2a) or (2b): 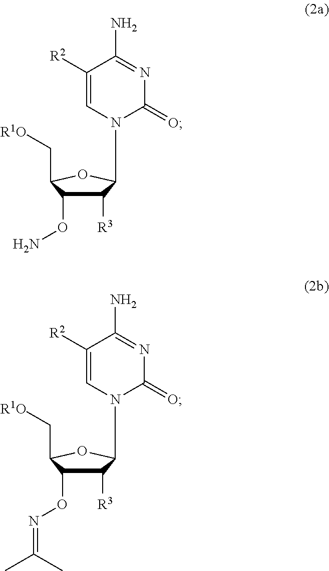
wherein,
R¹ is an oligonucleotide;
R² is an electron withdrawing group (EWG) selected from the group consisting of: halo; nitrile; halomethyl, dihalomethyl, trihalomethyl; C≡CR⁴; SOR⁴; SO₂R⁴; SO₃R⁴; COR⁴; CO₂R⁴; CONR⁴R⁵; and
R³ is selected from H, OH, F, OCH₃, or OCH₂CH₂OMe;
wherein R⁴ and R⁵ are independently selected from H and C₁₋₆ alkyl optionally substituted with OH or halo atoms.

19. The oligonucleotide according to claim 17 or 18, wherein R² is selected from the group consisting of: fluoro; propynyl and but-3-yn-1-ol.

20. The oligonucleotide according to claims 17 to 19, wherein R³ is H.

21. The oligonucleotide according to claims 17 to 20, wherein R⁶ is D.
